(19) United States
(12) Patent Application Publication
(10) Pub. No.: US 2003/0091999 A1
(43) Pub. Date: May 15, 2003

(54) Compositions and methods for identifying polypeptides and nucleic acid molecules

(76) Inventor: Zhongping Yu, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 09/821,160

(22) Filed: Mar. 29, 2001

Related U.S. Application Data
(63) A 371 of international application No. PCT/US00/26511, filed on Sep. 27, 2000.
(60) Provisional application No. 60/156,990, filed on Oct. 1, 1999.
(60) Provisional application No. 60/178,420, filed on Jan. 27, 2000.

(51) Int. Cl. C12Q 1/68; G01N 33/53; C07H 21/04

(52) U.S. Cl. 435/6; 435/7.1; 536/23.1

(57) ABSTRACT

The present invention provides efficient methods for identifying nucleic acid molecules and polypeptides encoded thereby. These methods can be performed using translation systems or methods. The result of these methods is a complex that binds its own encoding nucleic acid molecule. The complex preferably includes the polypeptide and nucleic acid molecule such that the nucleic acid molecule encodes at least a portion of the polypeptide.

This application claims benefit of priority to U.S. provisional application No. 60/156,990 which was filed on Oct. 1, 1999, which is incorporated herein by reference; to U.S. provisional application No. 60/178,420 which was filed on Jan. 27, 2000, which is incorporated herein by reference and to PCT application number PCT/US00/26511 which was filed on Sep. 27, 2000, which is also incorporated by herein by reference.

### TECHNICAL FIELD

The present invention relates generally to the fields of molecular biology, in particular to compositions and methods for identifying nucleic acid molecules and polyeptides.

### BACKGROUND

Efforts to identify polypeptides that have biological activity such as enzymatic activity or binding activity and the nucleic acid molecules that encode the polypeptide have utilized a variety of methods. Such methods include genomics and combinatorial biology.

Genomics generally identifies nucleic acid molecules within a genome, often without regard to the function of the nucleic acid molecule or the polypeptide encoded thereby. Genomics tends to provide information as to the sequence or partial sequence of a nucleic acid molecule but does not provide significant information as to the function of the nucleic acid sequence or the polypeptide encoded thereby. The outcome of genomics is generally the identification of expression sequence tags (ESTs) or the trapping of promoters or genes. Functional genomics generally attempts to contemporaneously identify a gene and its function. Functional genomics relies on the use of cell-based or organism-based assay systems or comparative analyses, which tend to be cumbersome, complicated, time-consuming and expensive.

Combinatorial biology generally identifies nucleic acid sequences and polypeptides encoded thereby that are not isolated from a biological source but nonetheless have a biological activity. Combinatorial biology provides random or semi-random groups (or libraries) of nucleic acid molecules or polypeptides. The libraries are screened for an activity, such as a binding activity. One method of combinatorial biology, known as SELEX, relies on the folding of RNA molecules to provide an RNA molecule that has receptor-ligand binding capabilities. This type of receptor-ligand binding, though interesting, is a rather rare event in cellular processes.

Another method of combinatorial biology provides a library of bacteriophages that display a variety of random polypeptides on their surface. The genome of the bacteriophage includes the nucleic acid molecule that encodes the random polypeptide displayed on the surface. The binding of a phage to a receptor during the “panning” procedure results in the isolation of a bacteriophage that includes the random polypeptide and the encoding nucleic acid molecule. This type of combinatorial biology results in the identification of interesting polypeptides and nucleic acid sequences, but the methods used rely on complex in vivo biological processes to produce bacteriophage. These complex processes tend to reduce the complexity of the combinatorial biology libraries and make these methods not particularly suitable for automation.

In vitro combinatorial biology methods have also been used. For example, random nucleic acid sequence can be made part of an RNA molecule that is translated by a plurality of ribosomes to form a polysome. The polysome structure can be “stalled” such that the RNA molecule is attached to the ribosomes and a partially translated polypeptide. This stalling can be accomplished using a variety of methods, such as incubating at low temperature or adding chemicals to stabilize the polypeptide-ribosome-RNA ternary structure. The stalled polysomes structures can then be panned for binding to a ligand to identify polysomes that include random nucleic acid molecules that encode polypeptides that can bind with a ligand. However, using the “stalled” ribosome complexes may limit the application of the method since the complex is not stable in many conditions. Also, these polysome display methods suffer from the formation of large, complex polysomes with a large number of polypeptides per RNA molecule. This is undesirable because the distribution of polysomes can be uneven, which can leave some RNA molecules free of ribosomes and therefore unselectable.

The present invention provided methods and articles of manufacture that address the problems associated with combinatorial biology. The present invention provides related benefits as well.

### BRIEF DESCRIPTION OF THE FIGURES

FIG. 1 depicts one aspect of a secondary structure in a nucleic acid molecule of the present invention.

FIG. 2 depicts a schematic diagram of one aspect of the present invention. MBR refers to moiety binding region, bFGF refers to fibroblast growth factor as an interacting domain, GST refers to glutathione-S-transferase as a spacer region and purification domain, and HA refers to a hemaglutinin tag sequence as a sequence of interest. “NAP” refers to a nucleic acid linked peptide, or complex of the present invention.

FIG. 3 depicts a schematic diagram of one aspect of the present invention. MBR refers to the binding moiety, ITD refers to interacting domain and RS refers to random sequence or sequence of interest. “NAP” refers to a nucleic acid linked peptide, or complex, of the present invention.

FIG. 4 depicts a schematic diagram of one aspect of the present invention in which transcription and translation are coupled, and a secondary structure can form in a transcribed RNA molecule. MBR refers to the binding moiety, ITD refers to interacting domain and RS refers to random sequence or sequence of interest.

FIG. 5 depicts a schematic diagram of one aspect of the present invention in which transcription and translation are coupled, the moiety binding region of the nucleic acid construct occurs 5′ of the ATG where translation initiates, and the interacting domain is downstream from the moiety binding region. MBR refers to the binding moiety, ITD refers to interacting domain and RS refers to random sequence or sequence of interest.

FIG. 6 depicts a schematic diagram of one aspect of the present invention. In this embodiment, the binding moiety is nitrilotriacetic acid (NTA), the interacting domain comprises six histidine residues, and the intermediary binding entity is nickel (Ni²⁺).

FIG. 7 depicts a schematic diagram of one aspect of the present invention. In this embodiment, the binding moiety is nitrilotricacetic acid (NTA), the interacting domain comprises six histidine residues, and the indirect binding entity is nickel (Ni²⁺).

### SUMMARY

The present invention provides a collection of efficient methods for identifying nucleic acids and polypeptides encoded thereby. These methods may be performed using translation systems or methods. The result of these methods is a complex that includes a polypeptide that binds with a nucleic acid molecule. The nucleic acid comprises or is operably linked to a binding moiety that directly or indirectly binds with an interacting domain encoded by at least a portion of said nucleic acid. Therefore the complex preferably includes the polypeptide and the nucleic acid molecule encodes at least a portion of the polypeptide.

A first aspect of the present invention is a nucleic acid molecule that 1) comprises of a binding moiety, and 2) encodes an interacting domain, wherein the interacting domain directly or indirectly binds with the binding moiety.

A second aspect of the present invention is a library of nucleic acid molecules of the present invention or complexes of the present invention, including libraries of nucleic acid molecules of the present invention or complexes of the present invention with a substance of interest or as part of a vector.

A third aspect of the present invention is a method for identifying a nucleic acid molecule.

A fourth aspect of the present invention is a method for identifying a polypeptide.

A fifth aspect of the present invention is a method of identifying a test compound and test compounds and pharmaceutical compositions identified by such methods.

A sixth aspect of the present invention is a method of identifying a target and targets and pharmaceutical targets identified by such methods.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, and cell biology and cell culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989); Ausubel et al., Current Protocols in Molecular Biology, John Wiley and Sons (1998) and Harlow and Lane, Antibodies, A Laboratory Manual, Cold Spring Harbor Press (1988)). Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Membrane permeant derivative” refers to a chemical derivative of a compound that increases membrane permeability of the compound. These derivatives are made better able to cross cell membranes because hydrophilic groups are masked to provide more hydrophobic derivatives. Also, the permeability-making groups can be designed to be cleaved from the compound within a cell to make the compound more hydrophilic once within the cell. Because the substrate is more hydrophilic than the membrane permeant derivative, it preferentially localizes within the cell (U.S. Pat. No. 5,741,657 to Tsien et al., issued Apr. 21, 1998).

“Isolated polynucleotide” refers to a polynucleotide of genomic, cDNA, or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, RNA derived from cDNA, DNA, RNA or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source (for example, free of cellular proteins), or (4) is expressed by a cell from a different species, or (5) does not occur in nature.

“Polypeptide” is used herein as a generic term to refer to native protein or fragments or analogs of a polypeptide sequence.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and which has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal biding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

“Polynucleotide” or “nucleic acid” or “nucleic acid molecule” refers to a polymeric form of nucleotides of a least ten bases in length, either ribonucleotides or deoxynucleotides or a modified from of either type of nucleotide. The term includes single and double-stranded forms of DNA, RNA or RNA/DNA hybrids.

“Directly” in the context of a biological process or processes, refers to direct causation of a process that does not require intermediate steps, usually caused by one molecule contacting or binding to another molecule (the same type or different type of molecule). For example, molecule A contacts molecule B, which causes molecule B to exert effect X that is part of a biological process.

“Indirectly” in the context of a biological process or processes, refers to indirect causation that requires intermediate steps, usually caused by two or more direct steps. For example, molecule A contacts molecule B to exert effect X which in turn causes effect Y.

“Sequence homology” refers to the proportion of base matches between two nucleic acid sequences or the proportion of amino acid matches between two amino acid sequences. When sequence homology is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonuleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and most preferably not less than 19 matches out of 20 possible base pair matches (95%).

“Selectively hybridize” refers to nucleic acid sequences that detectably and/or specifically bind. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence homology between the polynucleotides, oligonucleotides, and fragments thereof and a nucleic acid sequence of interest will be at least 30%, and more typically and preferably of at least 40%, 50%, 60%, 70%, 80% or 90%.

Hybridization and washing conditions are typically performed at high stringency according to conventional hybridization procedures. Positive clones are isolated and sequenced. For example, a full length polynucleotide sequence can be labeled and used as a hybridization probe to isolate genomic clones from an appropriate target library as they are known in the art. Typical hybridization conditions and methods for screening plaque lifts and other purposes are known in the art (Benton and Davis, Science 196:180 (1978); Sambrook et al., supra, (1989)).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at least 5 (in standard deviation units) using the program ALIGN with the mutation data matrix and a gap penalty of 6 or greater (Dayhoff, in Atlas of Protein Sequence and Structure, National Biomedical Research Foundation, volume 5, pp. 101-110 (1972) and Supplement 2, pp. 1-10). The two sequences or parts thereof are more preferably homologous if their amino acids are greater than or equal to 30% identical when optimally aligned using the ALIGN program.

“Corresponds to” refers to a polynucleotide sequence that is homologous (for example is identical, not strictly evolutionarily related) to all or a portion of a reference polynucleotide sequence, or to a polypeptide sequence that is identical to all or a portion of a reference polypeptide sequence. In contradistinction, the term “complementary to” is used herein to mean that the complementary sequence is homologous to or will base pair with all or a portion of a reference polynucleotide sequence. For illustration, the nucleotide sequence TATAC corresponds to a reference sequence TATAC and is complementary to a reference sequence GTATA.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a full length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local homology algorithm (Smith and Waterman, Adv. Appl. Math., 2:482 (1981)), by the homology alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of homology over the comparison window) generated by the various methods is selected.

“Sequence identity” means that two polynucleotide sequences are identical (for example, on a nucleotide-by-nucleotide basis) over the window of comparison.

“Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

“Substantial identity” as used herein denotes a characteristic of a polynucleotide sequence, wherein the polynucleotide comprises a sequence that has at least 30 percent sequence identity, preferably at least 50 to 60 percent sequence, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison.

“Substantial identity” as applied to polypeptides herein means that two peptide sequences, when optimally aligned, such as by the programs GAP or BESTFIT using default gap weights, share at least 30 percent sequence identity, preferably at least 40 percent sequence identity, and more preferably at least 50 percent sequence identity, and most preferably at least 60 percent sequence identity. Preferably, residue positions, which are not identical, differ by conservative amino acid substitutions.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; a group of amino acids having acidic side chains is aspartic acid and glutamic acid; and a group of amino acids having sulfur-containing side chan is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

“Modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

“Modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonists, partial antagonists, partial agonists, antagonists, antineoplastic agents, cytotoxic agents, inhibitors of neoplastic transformation or cell proliferation, cell proliferation promoting agents, antiviral agents, antimicrobial agents, antibacterial agents, antiprion agents, antiparasitic agents, antibiotics, and the like) by inclusion in assays described herein. Modulators can modulate the activity of biological processes in any type of cell, including prokaryotic cells and/or eukaryotic cells, such as, for example, plant cells, invertebrate cells, vertebrate cells, insect cells, mammalian cells and human cells, including cells derived from unicellular or multicellular terrestrial organisms, aquatic organisms or marine organisms The activity of a modulator may be known, unknown or partially known.

“Test compound” refers to a chemical, compound, composition or extract to be tested by at least one method of the present invention for at least one activity for at least one activity such as putative modulation of a biological process or specific binding capability. Test compounds can include small molecules, such as small molecules, drugs, proteins or peptides or active fragments thereof, such as antibodies or fragments or active fragments thereof, nucleic acid molecules such as DNA, RNA or combinations thereof, antisense molecules or ribozymes, or other organic or inorganic molecules, such as lipids, carboydrates, or any combinations thereof. Test compounds that include nucleic acid molecules can be provided in a vector, such as a viral vector, such as a retrovirus, adenovirus or adeno-associated virus, a liposome, a plasmid or with a lipofection agent. Test compounds, once identified, can be agonists, antagonists, partial agonists or inverse agonists of a target. A test compound is usually not known to bind to the target of interest. “Control test compound” refers to a compound known to bind to the target (for example, a known agonist, antagonist, partial agonist or inverse agonist). Test compound does not typically include a compound added to a mixture as a control condition that alters the function of the target to determine signal specificity in an assay. Such control compounds or conditions include chemicals that (1) non-specifically or substantially disrupt protein structure (for example denaturing agents such as urea or guandium, sulfhydryl reagents such as dithiothreitol and beta-mercaptoethanol), (2) generally inhibit cell metabolism (for example mitochondrial uncouplers) and (3) non-specifically disrupt electrostatic or hydrophobic interactions of a protein (for example, high salt concentrations or detergents at concentrations sufficient to non-specifically disrupt hydrophobic or electrostatic interactions). The term test compound also does not typically include compounds known to be unsuitable for a therapeutic use for a particular indication due to toxicity to the subject. Usually, various predetermined concentrations of test compounds are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test compounds, the concentration of test chemical used can be expressed on a weight to volume basis. Under these circumstances, the following ranges of concentrations can be used: between about 0.001 micrograms/ml and about 1 milligram/ml, preferably between about 0.01 micrograms/ml and about 100 micrograms/ml, and more preferably between about 0.1 micrograms/ml and about 10 micrograms/ml.

“Target” refers to a biochemical entity involved in a biological process. Targets are typically proteins that play a useful role in the physiology or biology of an organism. A therapeutic composition or compound typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, intracellular structures, tubules, tubulin, antibodies and the like.

A “therapeutic target” or a “pharmaceutical target” is a target that when modulated can have a therapeutic effect.

A “purification target” is a target that is useful in purification schemes, such as, for example, regions of antibodies such as the Fc region.

A “diagnostic target” is a target that is useful in diagnostics, such as cell surface epitopes or markers on etiological agents.

“Label” or “labeled” refers to incorporation of a detectable marker, for example by incorporation of a radiolabled compound or attachment to a polypeptide of moieties such as biotin that can be detected by the binding of a section moiety, such as marked avidin. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes such as ³H, ¹⁴C, ³⁵S, ³²P, ¹²⁵I or ¹³³I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

“Pharmaceutically effective amount” refers to an appropriate dose, regime, route of administration, time and delivery modality associated with the delivery of an amount of a compound or composition to cause a desired effect. Such pharmaceutically effective amount can be determined using methods described herein or by the United States Food and Drug Administration (USFDA).

“Sample” means any biological sample, preferably derived from a test animal, such as a mouse, rat, rabbit or monkey, or a patient, such as a human. Samples can be from any tissue or fluid, such as neural tissues, central nervous tissues, internal organs such as pancreas, liver, lung, kidney, muscle, skeletal muscle, urine, feces, blood, fluids from body cavities or the central nervous system, or samples from various body cavities such as the mouth or nose. Samples derived from urine and feces contain cells of the immunological, urinary or digestive tract and can be a rich source of sample. Such samples can be obtained using methods known in the art, such as biopsies, aspirations, scrapings or simple collection. A sample can be taken from a test animal or patient that is either living or dead.

“Ribozyme” means enzymatic RNA molecules capable of catalyzing the specific cleavage of RNA. The mechanism of ribozyme action involves sequence-specific hybridization of the ribozyme molecule to complementary target RNA, followed by endonucleolytic cleavage.

A “polynucleotide”, “nucleic acid” or “nucleic acid molecule” refers to any nucleic acid molecule, such as DNA or RNA, single-stranded or double-stranded, or modified polynuleotides such as those utilizing thiodiester bond in the backbones, or combinations thereof, such as single-stranded RNA forming a double helix with single-stranded DNA, herein referred to a “RNA/DNA hybrid”, or single-stranded DNA or double-stranded DNA operably covalently or non-covalently linked in a linear configuration with single-stranded RNA or double-stranded RNA, herein referred to as a “RNA/DNA chimera”. A nucleic acid molecule can comprise more than one form of nucleic acid, for example, it may be a molecule comprising both double-stranded and single-stranded RNA. A nucleic acid molecule can be a ribozyme. A nucleic acid molecule of the present invention includes a nucleic acid molecule with or without other components, such as a binding motif including a nucleic acid sequence or other chemical compound that binds an interacting peptide domain.

A “RNA/DNA hybrid” is a double-stranded nucleic acid molecule in which one strand consists of RNA and the other strand consists of DNA. RNA/DNA hybrids may be a part of nucleic acid molecules that comprises other forms of nucleic acids, such as double-stranded DNA, double-stranded RNA, single-stranded RNA, or single-stranded DNA.

A “RNA/DNA chimera” is a nucleic acid in which single-stranded or double-stranded DNA is operably covalently or non-covalently linked in a linear configuration with single-stranded or double-stranded RNA.

A “nucleic acid sequence” refers to the sequence of bases in a nucleic acid molecule, such as SEQ ID NO:1.

A “binding moiety” refers to a region of a nucleic acid molecule, a compound or an entity linked to a nucleic acid, that is able to bind directly or indirectly to a polypeptide domain encoded by a part of said nucleic acid. A binding moiety that is a region of a nucleic acid molecule may be referred to as a “moiety binding region”. For example, a RNA sequence that is a binding moiety can also be a moiety binding region that is able to bind with an interacting domain of a polypeptide. Another example of a binding moiety is nitrilotriacetic acid (NTA) that is covalently bound to the nucleic acid. The NTA is able to bind with a interacting domain, preferably a stretch of contiguous histidine residues, of a polypeptide that is encoded by said nucleic acid.

A “moiety” refers to any chemical or biochemical structure. Preferably, a moiety is a structure that can bind directly or indirectly with a polypeptide domain that results in a desired effect. A moiety is preferably a biochemical structure, such as a nucleic acid molecule, a lipid, a polypeptide, a carbohydrate, a small organic or inorganic compound (such as biotin and NTA), or a combination thereof.

An “interacting domain” refers to at least a portion of a polypeptide that directly binds or indirectly binds with the binding moiety of its own encoding nucleic acid.

An “intermediary binding entity” is a moiety that directly or indirectly binds the interacting domain and also directly or indirectly binds the binding moiety, thereby linking the interacting domain to the binding moiety. An example of an intermediary binding entity is nickel (Ni²⁺), which binds nitriloacetic acid, which can be a binding moiety, and also binds histidine, which may, in multimeric form, comprise the interacting domain.

“Bind” refers to any cohesive interactions between two molecules, which includes covalent bonds, coordinate bonds, hydrophobic interactions, electrostatic interactions, or a combination thereof.

“Directly binds” refers to a direct binding interaction between binding moieties. For example, X binds to Y without bridging moiety Z.

“Indirectly binds” refers to an indirect binding interaction between binding moieties. For example, X binds with Z and Z binds with Y, but X does not directly bind with Y or X cannot bind to Y without Z.

A “random sequence” refers to a fully random, partially random, or semi-random sequence of nucleic acid bases that forms a nucleic acid molecule or amino acids that form a polypeptide. Random sequences can be made using synthetic methods as they are known in the art, such as solid phase nucleic acid or solid phase polypeptide synthesis, or by enzymatic methods, such as polymerase reactions or digesting polypeptides or nucleic acids of natural or synthetic origin to obtain fragments thereof, or by any combination of these methods. Fully random refers to 1) sequences that have been made without statistical weight to the probability of inserting any one of the set of naturally-occurring bases or amino acids at a given position of the random sequence, or 2) sequences that have been made by fragmentation of at least one nucleic acid molecule. Semi-random refers to sequences that have been made with statistical weight as bases/amino acids and/or their sequence and can be made using synthetic methods known in the art or by digesting polypeptides or nucleic acid molecules (see, U.S. Pat. No. 5,270,163 to Gold et al., issued Dec. 14, 1993; and U.S. Pat. No. 5,747,253 to Ecker et al., issued May 5, 1998). Semi-random sequences can be nucleic acid or amino acid sequences that have been synthesized such that particular sequence combinations are preferred over other sequence combinations. For example, a semi-random nucleic acid sequence can be biased to preferetially include only a subset of the nucleic acid condons that encode particular amino acids, or can be biased such that the frequency of stop condons in the squence is reduced. Similarly, a semi-random nucleic acid or amino acid sequence can be synthesized such that, for example, condons for hydrophobic amino acids, or hydrophoci amino acids themselves, are less abundant in the sequence than would occur if the squence were totally random. Semi-random sequences can be made by directed chemical synthesis, and can, for example, be based on the synthesis of preferred condons that can be built into a multi-condon sequence as disclosed in PCT application US99/22436 (WO 00/18778) to Lohse et al., published Apr. 6, 2000, which is herein incorporated by reference. Partially random sequences are sequences that are in part known or identified sequences and are in part fully random or partially random sequences, and] can also be made by modifying or adding to identified or fixed sequences (Pasqualini and Ruoslahti, Nature 380:364-366 (1999); and U.S. Pat. No. 5,270,163 to Gold et al., issued Dec. 14, 1993).

A “sequence of interest” refers to a nucleic acid sequence or nucleic acid molecule that has been selected for by screening or otherwise identified. A sequence of interest can also be at least a portion of a known nucleic acid molecule or nucleic acid sequence. Preferably, an activity, such as an enzymatic activity or binding activity, of the amino acid sequence that can be partially or entirely encoded by the sequence of interest is known (but that need not be the case), and the sequence of interest includes sequences encoding at least one such activity or a portion of such activity. A sequence of interest can also be a nucleic acid molecule whose sequence that is known or not known, and can include in whole or in part at least a portion of a nucleic acid molecule derived from a cell, for example at least a potion of a cellular RNA or genomic DNA. A sequence of interest can be a sequence of at least a portion of a nucleic acid molecule directly isolated from one or more cells, or can be a sequence of at least a portion of a nucleic acid moleucle that has be cloned, reverse transcribed, or amplified from one or more nucleic acid molecules isolated from one or more cells.

A “spacer region” refers to a polypeptide sequence or a nucleic acid sequence that links a first polypeptide domain or nucleic acid sequence, and a second polypeptide domain or nucleic acid sequence, respectively. Appropriate polypeptide and nucleic acid spacer sequences are known in the art, such as flexible peptide regions of proteins. The polypeptide or nucleic acid spacer region is in the same polypeptide or nucleic acid chain, respectively, that the said first and second polypeptide domains or nucleic acid sequences reside. A polypeptide spacer region of the present invention preferably does not directly or indirectly interact with the polypeptide or nucleic acid of the present invention. Appropriate polypeptide spacer sequences are known in the art, such as alpha helix structures or unstructured regions. A spacer region can have additional functions, such as having a purification domain and/or a detection domain.

A “purification domain” refers to a polypeptide domain or nucleic acid sequence that has a property that allows molecules such as nucleic acid molecules or polypeptides or combinations thereof to be purified. For example, a purification domain can include an epifope or a unique nucleic acid sequence that allows a molecule to be isolated or purified using appropriate methods, such as immunoaffinity chromatography or nucleic acid hybridization methods.

A “detection domain” refers to a domain that can be detected, or has a function that can be detected. For example, a detection domain can include a nucleic acid sequence such as a unique nucleic acid sequence that can be directly or indirectly detected, or can include a nucleic acid sequence that encodes a peptide epitope sequence that can be recognized by a specific binding member such as an antibody or active fragment thereof, or that encodes a detectable enzymatic activity, preferably an activity detectably with a chromogenic, fluorogenic or luminescent substrate, such as, for example, horse radish peroxidase, beta galactosidase or luciferase. A detection domain can also include a nucleic acid sequence that encodes a protein that can be directly detected, such as green fluorescent protein, phycoerythrin, etc., that can be visually or spectrophotometrically detected.

“Substantially devoid of ribosomes” refers to the state of a nucleic acid molecule, such as an RNA molecule, that has a structure that results in fewer ribosomes being bound thereto if the structure were not present. This term can refer to individual nucleic acid molecules as well as populations of such molecules. “Devoid of ribosomes” means that no ribosomes are present on a nucleic acid molecule. This term can refer to individual nucleic acid molecules as well as populations of such molecules.

“Secondary structure” refers to a structure in a nucleic acid molecule that is more than the primary linear structure of the sequence of bases. Secondary structures can include a variety of configurations based at least in part on base-pairings, such as stem-loop configurations or hairpin configurations. (See, U.S. Pat. No. 5,270,163 to Gold et al., issued Dec. 14, 1993; and U.S. Pat. No. 5,747,253 to Ecker, issued May 5, 1998).

“Reduces the efficiency of translation” means that the efficiency of translation of that nucleic acid molecule is reduced as compared to the efficiency of translation of the nucleic acid molecule without a binding moiety, stem-forming sequence, or other structural features.

“Stem-forming sequence” is a sequence of bases in a nucleic acid molecule that is comprised of two half-stem sequences wherein the first half of the stem-forming sequence is able to base pair with the second half stem-forming sequence when the first and second stem-forming sequences are in single-stranded form. Stem-forming sequences may base-pair to form a double-stranded structure in a continuous strand of a nucleic acid molecule. Such double-stranded structures may be of any length, and may be a part of larger secondary structures of the nucleic acid molecule, such as stem-loop structures and hairpin structures, as they are known in the art.

“Binding moiety/interacting domain complex” or “complex” refers to a complex formed by the interaction of a binding moiety and an interacting domain. For example, a binding moiety on an RNA molecule can directly bind or indirectly bind with an interacting domain on a polypeptide to form a complex that comprises the binding moiety and the interacting domain.

“Substance of interest” refers to a compound that has been selected for screening or has been identified using the methods of the present invention.

“Solid support” refers to any solid support that can be used in a method of the present invention. Preferably, a solid support is used to immobilize a nucleic acid molecule of the present invention or a complex of the present invention. In addition, a solid support can be used to immobilize a substance of interest, a cell, an etiological agent, or other moiety. Solid substrates can take any form, such as sheets, membranes (such as nitrocellulose or nylon), wells (such as microtiter wells), beads or microparticles, or chips, such as glass, nylon, or silica sheets that comprise arrays of nucleic acids, proteins, or other molecules. Solid supports can be of any appropriate material, such as polymers, metals, glass, or silica and can be magnetic in nature. Preferred solid substrates include polystyrene, polycarbonate, latex, polyacrylamide, nylon, nitrocellulose, glass, silica, and magnetite.

“On or within a cell” refers to a moiety, such as a receptor or biomolecule that resides on the surface of a cell, within the outer membrane of a cell, or within a cell. Within a cell refers to any locus within a cell, such as in the cytoplasm or within or associated with an organelle, such as, for example, a mitochondria, nucleus or golgi apparatus.

A “cell” refers to any cell, such as a of prokaryotic (such as bacterial) or eukaryotic origin. Eukaryotic cells include, for example, single cell organisms such as yeast and multicellular organisms such as invertebrates, plants and vertebrates. Invertebrates include parasites such as worms and vertebrates include cold-blooded organisms (such as reptiles and amphibians) and warm-blood organisms, such as mammals, including humans. A cell can be part of a sample of tissue, fluid or organ of a multicellular organism, or can be part of a multicellular organism itself.

“In vitro” refers to procedures that are performed outside of a cell. For example, purified enzymes or extracts of cells can be used to perform procedures in a vessel, such as a test tube.

“Ex vivo” refers to procedures that are performed outside of a multicellular organism, but use whole cells. For example, live cells from a subject, such as a human, can be cultured outside of the body and these cells can be used in testing procedures.

“In vivo” refers to procedures that are performed on a whole organism, such as a subject, including a human, such as in clinical trials. In vivo procedures can also be performed on non-human subjects, such as animal models.

A “normal cell” refers to a cells whose processes and characteristics are in conformance with an average cell of that type. For example, a normal lung cell does not exhibit the proliferation and metastatic capabilities of a cancerous lung cell.

An “abnormal cell” refers to a cell whose processes and characteristics are not in conformance with an average cell of that type. For example, a normal CD4+ does not exhibit the lifespan of a CD4+ cell infected with a virus, such as HIV.

A “neoplastic cell” refers to a cell that exhibits the processes and characteristics of a neoplasm, such as tumors, cancers, carcinomas and the like.

A “virus infected cell” refers to a cell that has been infected with a viable virus and exhibits or will exhibit characteristics of that infection.

An “etiological agent” refers to any etiological agent, such as bacteria, parasites, fungi, viruses, prions and the like.

A “library” refers to a group of two or more compounds or compositions. The members of a library can be mixed into a single population, such as in a single container. Alternatively, the members of a library can be provided separately in different containers, such as in microtiter plates or separate containers in a larger container, such as vials in a box. Alternatively, such separate containers can include one or more members of a library.

Other technical terms used herein have their ordinary meaning in the art that they are used, as exemplified by a variety of technical dictionaries, such as the McGraw-Hill Dictionary of Chemical Terms and the Stedman's Medical Dictionary.

Introduction

As a non-limiting introduction to the breath of the present invention, the present invention includes several general and useful aspects, including:

1) a nucleic acid molecule that comprises a binding moiety and that encodes an interacting domain, wherein the interacting domain directly or indirectly binds with the binding moiety;

2) a library of nucleic acid molecules of the present invention, either alone or with a substance of interest or as part of a vector;

3) a method for identifying a nucleic acid molecule;

4) a method for identifying a polypeptide that can be used in a variety of applications, such as diagnostics, affinity purification and therapeutics;

5) a method of identifying a test compound and test compounds and pharmaceutical compositions identified by such methods; and

6) a method of identifying a target and targets and pharmaceutical targets identified thereby.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### I Nucleic Acid Constructs

The present invention includes nucleic acid molecules in the form of constructs that are useful for a variety of purposes, including methods of the present invention. The nucleic acid constructs can be provided in vectors, can comprise binding moieties, and can have additional characteristics such as secondary structure. The nucleic acid molecules of the present invention can be made using any appropriate method, including synthetic methods or cloning methods as they are known in the art (Sambrook et al., supra, (1989)). Nucleic acid molecules can be made using a combination of methods, including chemical synthesis, PCR amplification, restriction digestion, ligation, polymerization, reverse transcription, etc. and can use nucleic acid molecules, templates, and sequences of from any sources, including identified nucleic acid molecules, genomic DNA, cellular RNA, etc.

A nucleic acid molecule of the present invention preferably comprises a binding moiety or comprises sequences encoding a binding moiety, such as a DNA molecule that encodes an RNA binding moiety, and encodes an interacting domain. Preferably, the interacting domain directly or indirectly binds with the binding moiety. The nucleic acid molecule can be any nucleic acid molecule, but is preferably double-stranded DNA or single-stranded RNA, and can be single-stranded DNA or double-stranded RNA, and can also be a RNA/DNA hybrid molecule, or a RNA/DNA chimera. A nucleic acid of the present invention may comprise these forms of nucleic acid in any combination, for example, a nucleic acid of the present invention may comprise both single-stranded and double-stranded RNA. Binding moieties can be a part of a nucleic acid molecule, including DNA or RNA, or any compounds that are bound to the nucleic acid; whereas interacting domains are part of a polypeptide that are encoded by DNA or RNA.

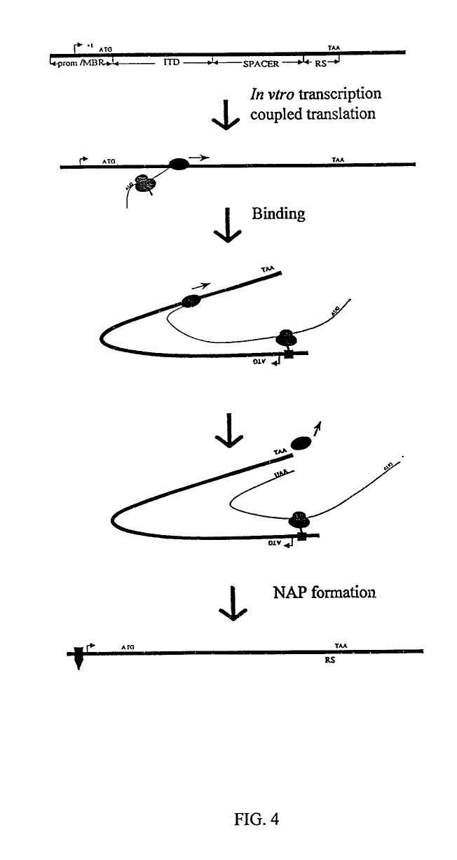

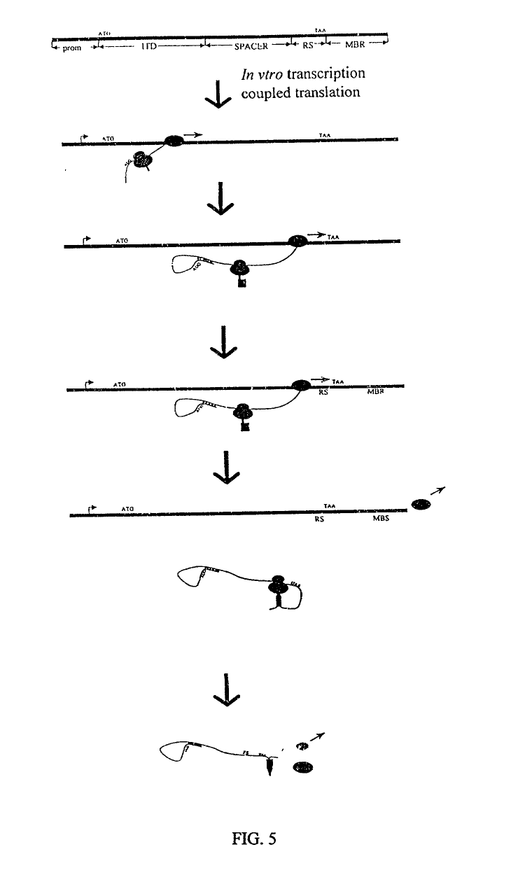

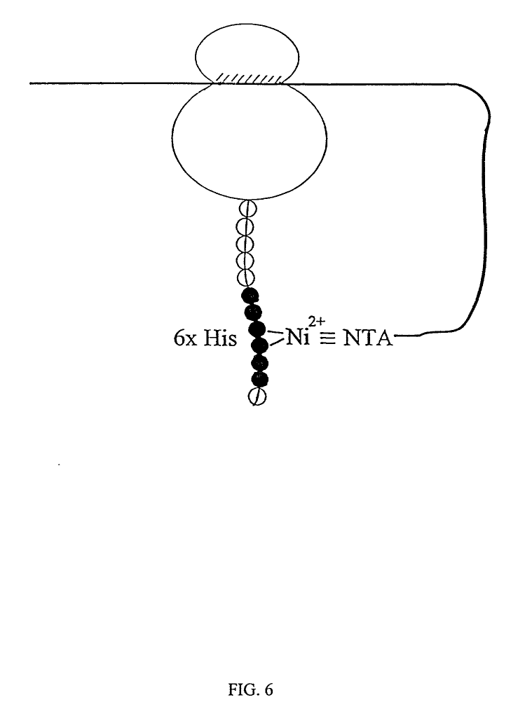

Binding moieties are regions of nucleic acid molecules or are chemical compounds covalently bound to nucleic acid molecules that can bind with the interacting domain. Preferably, binding moieties are part of an RNA molecule (see FIG. 4), but may also be part of a DNA molecule (see FIG. 5), or may be a compound covalently attached to a nucleic acid molecule, such as, for example, biotin attached to a DNA molecule or the present invention, or nitrilotriacetic acid attached to an RNA molecule of the present invention (see FIG. 6). Binding moieties that are part of a nucleic acid molecule of the present invention can be encoded by DNA and have function in an RNA molecule the DNA encodes, or binding moieties can have function as part of a DNA molecule. For example, regions in an RNA can have the function of binding a polypeptide. That function may or may not be attributable to positive or negative sense single strands of DNA that gave rise to the mRNA region via transcription. A binding moiety can also directly bind with an intermediary binding entity that allows an interacting domain to indirectly bind with a binding moiety. Binding moieties, intermediary binding moieties and interacting domains can be selected based on their expected properties as they are known in the art. The length of nucleic acid sequence or amino acid sequence that relate to the function of these structures can be selected based on reports in the literature, or by screening portions of such sequences for the desired activity using standard binding assay methods as they are known in the art.

The binding moiety and the sequence encoding the interacting domain need not be directly linked together, immediately adjacent to each other or be part of the same nucleic acid molecule, but are preferably operably linked. These elements of the nucleic acid molecule of the present invention can be provided on a nucleic acid construct in any order or orientation.

In one embodiment of the present invention, exemplified in FIG. 4, the nucleic acid molecule is a double-stranded DNA and comprises a sequence at the 3′-end that, when transcribed into RNA, serves as the binding moiety. Preferably, the RNA transcribed from the double-stranded DNA construct further comprises RNA stem-forming sequences at the 5′ end that comprise two half-stem-sequences that are between 4 and 100 bases in length, and are complementary to one another. The first half-stem-sequence is preferably in close proximity to the 5′ end of the RNA transcript encoded by the DNA molecule and the second half-stem-sequence is preferably downstream of the first half-stem-sequence, and the two half-stem-sequences are separated by a sufficient number of base pairs to allow base pairing of the two sequences when they are transcribed into RNA. The first half-stem sequence is preferably within one to 50 bases from the 5′ end of the RNA transcript encoded by the DNA molecule and preferably the second half-stem sequence is between 3 and 2000 bases downstream of the first half-stem sequence. Base pairing of the stem-forming sequences of the transcribed RNA preferably reduces the efficiency of translation of the RNA. Preferably, in methods using nucleic acid constructs that include stem-forming sequences at the 5′ end, translation of the RNA is coupled to transcription of the DNA, such that a ribosome can bind the RNA while transcription of the DNA into RNA is progressing and before the secondary structure of the stem-forming sequence forms, whereas the efficiency of subsequent translation events are reduced by the formation of secondary structure by the stem forming sequence. In this way, only one or a few peptides are translated from each RNA. A variety of nucleic acid constructs of the present invention can employ stem forming sequences. In embodiments of the invention that include stem forming sequences, the binding moiety is preferably at the 3′ end of the nucleic acid, and the sequences encoding the interacting domain may be anywhere 5′ of the binding moiety, such that there is sufficient spacing that the interacting domain may interact with the binding moiety subsequent to translation of the interacting domain. The interacting domain is preferably at the 5′ end of the construct, following the stem-forming sequence and translational start site.

Nucleic acid molecules of the present invention can be of any length, but are preferably between about 50 bases and about 10,000 bases, more preferably between about 100 bases and about 2,000 bases in length. If the binding moiety is a part of the nucleic acid molecule, it is preferably between about 8 bases and about 1,000 bases, more preferably between about 12 bases and about 150 bases in length. The interacting domain is preferably between about 1 amino acid and about 1,000 amino acids, more preferably between about 2 amino acids and about 150 amino acids in length, which is encoded by a nucleic acid molecule of appropriate length.

A nucleic acid molecule of the present invention can further include at least one random sequence or at least one sequence of interest or a combination of at least one random sequence and at least one sequence of interest. The random sequence or sequence of interest can be made using appropriate methods in the art, such as cloning techniques, including PCR techniques, enzymatic techniques such as reverse-transcription from cellular mRNA or polymerases such as terminal transferases, solid phase synthesis or fragmenting nucleic acid molecules using a variety of methods, such as sheer forces, vibrational energy, chemical agents, or restriction enzymes, or a combination of these methods. For the random sequences from synthetic origins, the polynuleotides can be of any length, but are preferably between about 10 bases and about 500 bases, more preferably between about 30 bases and about 120 bases in length.

Fully random sequences can be chemically synthesized, or generated by fragmentation of a complex nucleic acid molecule or a population of nucleic acid molecules (such as a chromosome or genome) and optionally amplified (including the use of amplification methods using conditions that favor random incorporation or misincorporation), and/or can have additional random, semi-random, or nonrandom sequences added to them (such as, for example, by terminal transferase, ligation, or chemical conjugation.

Partially random sequences can be acheived by producing a random oligomer by chemical synthesis and inserting it into a sequence of interest, such as a sequence of a known protein, to create mutations as described in Ausubel et al., Current Protocols in Molecular Biology, John Wiley and Sons, 1994). Other methods of mutagenizing include polymerase reactions that allow for misincorporation of nucleotides, site directed mutagenesis, or template directed mutagenesis (Ausubel et al., supra; Sambrook et al., supra; Stemmer (Nature 370: 389 (1994); and Joyce and Inoue, Nucl. Acids Res. 17: 171 (1989)). Partially random sequences can also be created using a combination of techniques, for example, using template mutagenesis (where the template can be one or more identified or unidentified nucleic acid molecules, including DNA molecules from cellular sources or virus, or from libraries) followed by primed synthesis of nucleic acid segments of template as described in WO 00/00632, PCT application PCT/US99/14776, herein incorporated by reference.

Semi-random sequences can be made by chemical synthesis in which the base mixtures used contain different proportions of each base, or by controlled synthesis in which the addition of each base in a sequence is controlled such that, for example, only a subset of codons is synthesized (see, for example, PCT application US99/22436 (WO 00/18778) to Lohse et al., published Apr. 6, 2000).

A sequence of interest can be any nucleic acid sequence whose sequence is known, and whose identity may be known or unknown, or is at least a portion of a nucleic acid molecule derived from a cell, for example at least a portion of a cellular RNA or genomic DNA. For sequences of interest from biological origins such as those derived from mRNAs encoding antibodies or families of receptors or enzymes, the nucleic acid can be of any length, preferably between about 100 bases and 10,000 bases, more preferably between about 200 bases and 2,000 bases. A sequence of interest can include a nucleic acid molecule that is a member of a library, such as a cDNA library or genomic library, can be a cellular or viral RNA or a cDNA molecule obtained from reverse transcription of cellular or viral RNA, or a fragment thereof, or can be a fragment of a genomic or organellar DNA. The sequence of the sequence of interest may be known or unknown. The identity of the sequence of interest may or may not be known. If the sequence of interest is known, it can be made by chemical synthesis or enzymatic and cloning techniques, or combinations thereof, and incorporated into a nucleic acid molecule of the present invention. If the sequence of interest is obtained directly or indirectly from cellular RNA or DNA, it can be optionally cloned, amplified, or fragmented before incorportation into a nucleic acid molecule of the present invention.

When the source of a sequence of interest is a library, the sequence of interest can optionally be positioned 3′ of the sequence encoding the interacting domain, such that the potential occurrence of a termination codon in the sequence of interest does not disrupt the translation of the sequence encoding the interacting domain. Alternatively, techniques may be employed to avoid the occurrence of termination codons in a sequence of interest, for example, where the sequence of interest is obtained from a population of cDNA, the 3′ end can be removed, for example, using methods disclosed in WO 09737, PCT application PCT/US99/18603, herein incorporated by reference.

The binding moiety, the sequence encoding the interacting domain and the random sequence or sequence of interest need not be directly linked together, immediately adjacent to each other or provided on the same nucleic acid molecule, but are preferably operably linked. These elements of the nucleic acid molecule of the present invention can be provided on a nucleic acid construct in any order or orientation, but preferably that portion of the nucleic acid molecule encoding the interacting domain and random sequence or sequence of interest is 5′ to the spacer region. The binding moiety can be 5′ to the sequence encoding the interacting domain or 3′ to the random sequence or sequence of interest. The nucleic acid molecules of the present invention can be made using any appropriate method, including synthetic methods or cloning methods as they are known in the art (Sambrook et al., supra, (1989)).

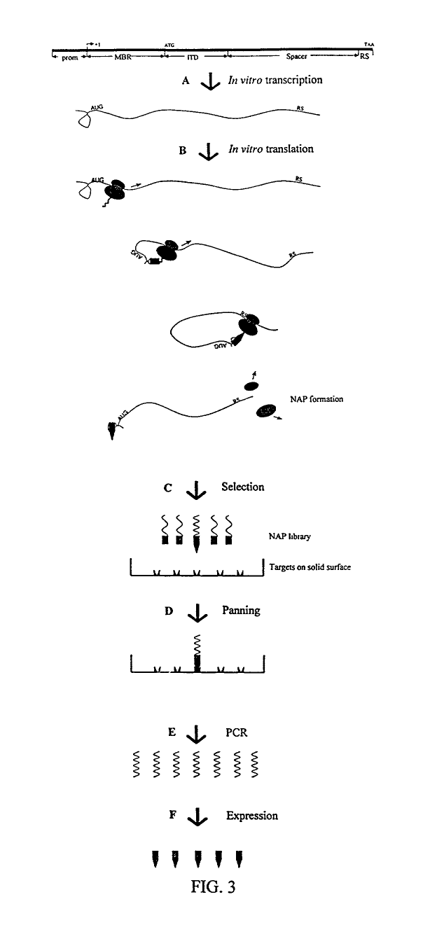

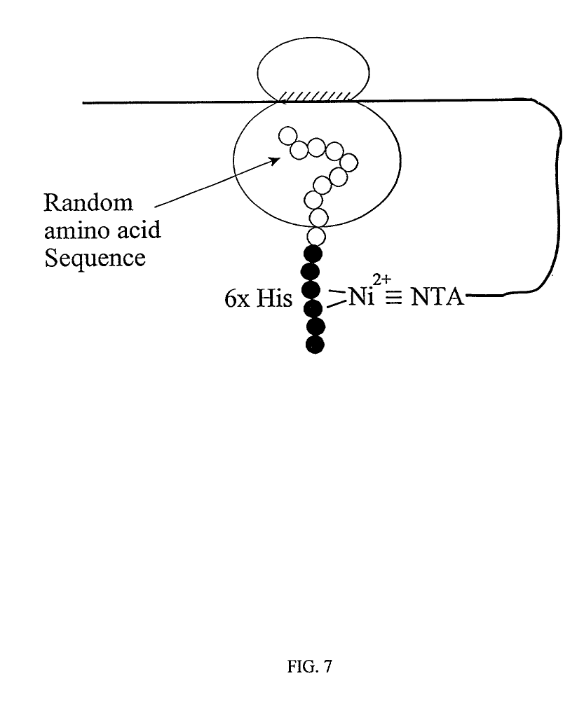

The nucleic acid molecule of the present invention can further include sequences that function as or encode at least one spacer region. The spacer region includes nucleic acid sequences including nucleic acid sequences encoding polypeptides that preferably do not interact with nucleic acids. The spacer region can be made using appropriate methods in the art, such as solid phase synthesis or cloning methods known in the art. The spacer region can be of any length, but is preferably between about 10 bases and about 1,000 bases, more preferably between about 30 bases and about 200 bases in length. Preferred spacer regions include sequences encoding glutathione-S transferase (GST) or green fluorescent protein (GFP) (see FIG. 3). The spacer region can include at least one purification domain or at least one detection domain. Preferred purification domains include, for example, GST and preferred detection domains include, for example, GFP. A distinct spacer region is not a requirement of the present invention. For example, in one of the preferred embodiments, illustrated in FIG. 7, the construct comprises a 5′-end binding moiety linked to sequences encoding the interaction domain, which are linked to a random sequence.

The binding moiety, the sequence encoding the interacting domain, the random sequence or sequence of interest and spacer region need not be directly linked together, immediately adjacent to each other or provided on the same nucleic acid molecule, but are preferably operably linked. These elements of the nucleic acid molecule of the present invention can be provided on a nucleic acid construct in any order or orientation. The binding moiety can be anywhere in the nucleic acid, which can be 5′ to the sequence encoding the interacting domain or 3′ to the random sequence of sequence of interest. The nucleic acid molecules of the present invention can be made using any appropriate method, including synthetic methods or cloning methods as they are known in the art (Sambrook et al., supra, (1989)) and described in the previously.

The nucleic acid molecules of the present invention may further include sequences that encode peptides that mediate the entry of peptides and other molecules into cells. Such sequences include sequences that encode portions of the tat gene of HIV (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999)) and portions of the Drosophila Antennapedia gene (Derossi, et al. J. Biol. Chem. 269: 10444-10450 (1994)) and other sequences that encode peptides that mediate entry of proteins into cells as they are known or become known in the art. Furthermore, the nucleic acids of the present inventions may further include sequences that encode peptides that direct molecules to particular cellular compartments, for example, the endoplasmic reticulum or the mitochondria, as such sequences are known in the art or are later identified or developed.

The nucleic acid molecule of the present invention preferably include at least one control sequence, such as an expression control sequence, that drives or regulates the transcription and/or translation of the nucleic acid molecule of the present invention. Sequences regulating transcription are well known in the art, such as, for example the CMV promoter for eukaryotic transcription systems, and T7, T3, and SP6 promoters for prokaryotic transcription systems. In embodiments of the present invention where constructs of the present invention are introduced into cells, inducible or tissue specific promoters can be optionally be used. For translation, preferred control sequences are “Kozak sequences” (Kozak, J. Biol. Chem. 266:19867-19870 (1991)) or IRES sequences, and at least one start codon and optionally at least one stop codon. However, in some applications, the nucleic acid may not include such control sequences.

The nucleic acids of the present invention may also include sequences that enhance the stability of RNAs in cells, such as sequences from the 3′ UTR of the tau gene (Aronov et al., J. Mol. Neurosci. 12: 131-145 (1999)) or the GAP43 gene (Kohn et al., Brain Res. Mol Brain Res. 36: 240-250 (1996)) that can extend the half-life of an mRNA. Such RNA stability sequences are preferably at the 3′ end of the nucleic acids of the present invention. RNA stability sequences to be included in the nucleic acid molecules may be cell-type specific, and may be chosen based on the cell type into which the nucleic acid molecule or complex of the present invention may be introduced.

The nucleic acid molecules of the present invention can optionally directly or indirectly labeled with a detectable marker. The detectable marker may be a radioisotope or a nonradioactive detectable molecule such as biotin, or other detectable markers as they are known or developed in the art. The marker may be directly or indirectly bound to the nucleic acid.

Constructs in Cells

A nucleic acid molecule of the present invention and complexes that include such nucleic acid molecules can also be provided in a cell. The nucleic acid molecule can be introduced into a cell using methods known in the art, such as lipofection or electroporation. In addition, nucleic acid molecules can be introduced into cells using vectors, such as viruses or phages. The cells can be any cells, including prokaryotic or eukaryotic cells, and can be ex vivo or in vivo, including within a whole organism, including a mammal, including a human. Once introduced into a cell, a nucleic acid molecule can be transcribed and/or translated to produce a complex of the present invention. In embodiments where a nucleic acid molecule of the present invention comprises one or more structures that reduces the efficiency of translation, a dosage effect can be acheived in the cells in which the construct is expressed.

Constructs with Linear Nucleic Acid

The nucleic acid molecules of the present invention, including those that optionally include a sequence of interest or a random sequence, can also be provided in the linear form of nucleic acid molecule. If the nucleic acid provided is single-stranded, the nucleic acid can be preferably translated to polypeptide directly. If the nucleic acid molecule provided is double-stranded, the nucleic acid must be converted to single-stranded form using the methods known in the art, and then translation can be carried out.

Constructs in Vectors

The nucleic acid molecules of the present invention, including those that optionally include a sequence of interest or a random sequence, can also be provided in a vector. Vectors can be viral vectors, liposomes, microspheres, plasmids, phages or linear dsDNA molecules. Vectors preferably include double-stranded DNA molecules of the present invention, but the invention is not limited to such vectors. For example, various viral vectors include single-stranded DNA (parvoviruses), single-stranded RNA (retroviruses) or double-stranded RNA (rotaviruses). Vectors are useful for making libraries of nucleic acid molecules of the present invention, particularly libraries that include nucleic acid molecules that have different random sequences or sequences of interest. Furthermore, such vectors are convenient for making, storing and transporting nucleic acid molecules of the present invention. Vectors can be made or modified using methods in molecular biology as they are known in the art (Sambrook et al., supra, (1989)). The vector of the present invention can be of any vector as that term is known in the art.

Vectors can be any vector known in the art, for example, retroviruses (U.S. Pat. No. 5,399,346 to Anderson et al., issued Mar. 21, 1995, Bandara et al., DNA and Cell Biol. 11:227-231 (1992)); adenoviruses (Berkner, BioTechniques 6: 616-629 (1989); adeno-associated viruses (Larrick and Burck, Gene Therapy. Application of Molecular Biology, Elsevier, New York (1991); plasmid vectors (U.S. Pat. No. 5,240,846 to Collins et al., issued Aug. 31, 1993); liposomes (Holmberg et al., J. Liposome Res. 1:393-406 (1990) and Liu et al., Nature Biotechnology 15:167-173 (1997)); microspheres (Mathlowitz et al., Nature 386:410-(1997)); see generally Larrick et al., Gene Therapy, Elsevier, New York (1991) and Pinkert, Transgenic Animal Technology, Academic Press, San Diego (1994).

The present invention includes a cell that includes or has been transfected or transformed by a vector of the present invention. Such a cell can be ex vivo or in vivo in a subject, including a test animal or a human. Preferably, the nucleic acid molecule of the present invention in the vector can be expressed in the cell. In one aspect of the present invention, the nucleic acid molecule includes a sequence of interest such that when translated retains at least one activity of the polypeptide encoded by the sequence of interest. Alternatively, the vector includes at least one random sequence. The activity of the polypeptide encoded by the random sequence in the cell can be monitored by observing or interrogating the cell using methods known in the art, including the use of reporter genes to report changes in signal transduction within a cell.

Constructs with Binding Peptides

The nucleic acid molecule of the present invention can also be operably linked to the interacting domain. Preferably, the operable link between the nucleic acid molecule of the present invention occurs with the direct binding or indirect binding of the binding moiety with the interacting domain. Preferably, the binding moiety is part of a nucleic acid molecule that also encodes the interacting domain. A ribosome translates the sequence encoding an interacting domain into a polypeptide and the translated polypeptide that includes the interacting domain binds with the binding moiety. Preferably, it is the interacting domain translated from a particular nucleic acid molecule that binds with that same nucleic acid molecule, but that needs not be the case.

The interactions involved in the direct binding or indirect binding of the binding moiety to the interacting domain can be any interactions that result in a reversible binding or irreversible binding. Irreversible binding is characterized by covalent or co-ordinate linkages as they are known in the art. Such irreversible binding can be made using cross linking agents, such as gluteraldehyde or UV irradiation, or the binding can be catalyzed, such as by by ribozyme-protein interaction. Reversible binding is characterized by short-range interactions, such as ionic interactions, hydrophobic interactions, hydrogen bonding, co-ordinate binding and Van der Walls interactions.

In this aspect of the present invention, the binding of the binding moiety to the interacting domain may result in a structure that reduces the efficiency of further ribosome binding to the mRNA molecule, such that the nucleic acid is substantially devoid or devoid of ribosomes, but that is not a requirement of the present invention. Also, the nucleic acid molecule can adopt a particular structure itself that can reduce the efficiency of further translation. In aspects of the present invention where constructs of the present invention are introduced into cells (ex vivo, in vitro, or in vivo), this can result in a dosage effect of expression of a sequence of interest or random sequence of a construct of the present invention.

Constructs with Random Peptides or Sequences of Interest

A nucleic acid molecule of the present invention can also be operably linked to a polypeptide encoded by a random sequence or a sequence of interest. In this aspect of the present invention, the binding moiety of the nucleic acid molecule can directly or indirectly bind with the interacting domain, so that the nucleic acid is linked to the polypeptide encoded by that nucleic acid molecule that also encodes the peptide encoded by the random sequence or sequence of interest. The interacting domain can be operably linked to the polypeptide encoded by the random sequence or sequence of interest.

Operably linked in this instance refers to the case where the interacting domain can directly or indirectly bind with the binding moiety and the polypeptide encoded by the random sequence or sequence of interest is capable of binding with a substance of interest, such as a ligand. Operably linked preferably includes the function of the binding of the binding moiety to the interacting domain that can also result in a structure that reduces the efficiency of further ribosomal binding to the nucleic acid molecule, such that the nucleic acid is substantially devoid or devoid of ribosomes and/or reduces the efficiency of the further translation of the mRNA molecule, but this is not a requirement of the present invention.

Complexes comprising nucleic acid molecules of the present invention operably linked to polypeptides may be labeled with a detectable label. The detectable label may be a radioisotope or a nonradioactive detectable molecule such as biotin, or other detectable moieties as they are known or developed in the art. The label may be directly or indirectly bound to the nucleic acid of the complex or to a polypeptide of the complex, or both. The polypeptide of the complex labeled with a detectable marker need not be the polypeptide encoded by or partially encoded by the random sequence or sequence of interest.

Constructs with or without Secondary Structure

In one aspect of the present invention, the nucleic acid molecule of the present invention includes a binding moiety that can be substantially free of secondary structure while maintaining the function of directly binding or indirectly binding the interacting domain. Preferably, the direct binding or indirect binding of the binding moiety with the interacting domain directly or indirectly reduces the efficiency of further ribosomal binding to the nucleic acid molecule, results in a nucleic acid molecule substantially devoid or devoid of ribosomes and/or reduction of the efficiency of the further translation of the nucleic acid molecule, but that is not a requirement of the present invention.

In another aspect of the present invention, the nucleic acid molecule of the present invention includes at least one secondary structure, such as, for example, a stem-loop configuration or a hairpin configuration or two stretches of complimentary sequences on the nucleic acid molecule. Preferably, the secondary structure is in close proximity (upstream, downstream or integral to) to a start codon. Preferably, the secondary structure is within between about 60 nucleotides and about 2 nucleotides of a start codon, within between about 50 nucleotides and about 4 nucleotides of a start codon, within between about 40 nucleotides and about 6 nucleotides of a start codon, within between about 30 nucleotides and about 8 nucleotides of a start codon or within between about 20 nucleotides and about 10 nucleotides of a start codon.

The secondary structure, either alone or in combination with the locus of the start codon, preferably directly or indirectly reduces the efficiency of further ribosomal binding to the mRNA molecule, resulting in a mRNA that is substantially devoid or devoid of ribosomes and/or reduces the efficiency of the translation of the mRNA molecule.

Binding Moiety Plus Interacting Domain

The nucleic acid molecule of the present invention can form a structure where the binding moiety and the interacting domain form a binding moiety/interacting domain complex. The binding moiety and the interacting domain can directly bind or indirectly bind. Preferably, the binding moiety is a part of an RNA or molecule that encodes that interacting domain that binds with the binding moiety. Also preferably, the binding moiety can be a compound that is bound to the nucleic acid, for example, the binding moiety can be biotin that is bound to a DNA molecule of the present invention, and the interacting domain can comprise at least a portion of the avidin protein. The binding moiety and interacting domain are operably linked such that their binding can preferably directly or indirectly reduce the efficiency of further ribosomal binding to the mRNA molecule, resulting in a mRNA substantially devoid or devoid of ribosomes, but that is not a requirement of the present invention. Preferably, binding of the binding moiety to the interacting domain reduces the efficiency of the translation of the mRNA molecule, but that is not a requirement of the present invention. In the alternative, where the nucleic acid molecule is a double-stranded DNA molecule, binding of the interacting domain to the binding moiety may reduce the efficiency of transcription of the double-stranded DNA nucleic acid molecule (see, for example, FIG. 5).

Construct Bound with a Substance of Interest

In another aspect of the present invention, a nucleic acid molecule of the present invention can form a structure where the polypeptide encoded by a random sequence or sequence of interest is bound with a substance of interest. This aspect of the invention allows the selection of polypeptides that bind with a substance of interest, while at the same time selecting the nucleic acid molecule that encodes the polypeptide that binds with a substance of interest. The substance of interest can be on a solid support, and can be immobilized on such a solid support using methods known in the art such as absorption, chemical conjugation or cross-linking.

Alternatively, the structure formed by a nucleic acid of the present invention and a polypeptide encoded by a random sequence or sequence of interest can be immobilized on a solid support and one or more substances of interest may be bound with, or capable of reacting with, the fixed complex or complexes.

Furthermore, the substance of interest can be on or within a cell, and the cell can be immobilized on a solid support using appropriate methods, such as solid supports covered with fibronectin or other adhesion molecules. The cell can be ex vivo and can be provided as a cell, culture of cells, or part of a sample of tissue, fluid or organ. Alternatively, the cell can be in vivo in a subject.

The substance of interest may be a cell-type-specific or tissue-specific molecule, such that nucleic acids or peptides of the present invention that specifically bind to the substance of interest can be identified. Peptides of the present invention that specifically bind cell-type-specific and tissue-specific molecules can be used to target drug delivery to specific cells or tissues.

The cell can be any cell, including a normal cell or an abnormal cell, such as, for example, a neoplastic cell or a virus infected cell. The substance of interest can also be on or within an etiological agent, such as, for example, a virus, a bacteria, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest can also include at least one organic molecule, an inorganic molecule, a polymer, a polypeptide, a lipid or steroid, a carbohydrate, a small molecule, a nucleic acid molecule, a ribozyme, a biomacromolecule or a drug.

### II Libraries

The present invention also includes a library of nucleic acid molecules of the present invention. Such libraries include nucleic acid molecules with or without additional moieties, such as, for example, interacting domains that can be bound with a binding moiety of a nucleic acid molecule and/or a moiety that allows an interacting domain to indirectly bind with a binding moiety.

The library of nucleic acid molecules can include at least two different random sequences, at least two different sequences of interest or a combination of at least one random sequence and at least one sequence of interest. For example, a library of nucleic acid molecules can include two such nucleic acid molecules. Each nucleic acid molecule can have a different random sequence or a different sequence of interest. In addition, one nucleic acid molecule can have a random sequence and the other nucleic acid molecule can have a sequence of interest.

The library can be fixed to a solid support. In particular, libraries containing random sequences, which may include sequences in which one or a plurality of sequence positions have been randomly or semi-randomly varied, or libraries containing sequences of interest, can be fixed to a chip or array for screening with one or more substances of interest. A translated library of complexes may also be fixed to a solid support. In particular, libraries of complexes containing random sequences, which may include sequences in which one or a few sequence positions have been randomly or semi-randomly varied, or libraries of complexes containing sequences of interest, can be fixed to a chip or array for screening with one or more substances of interest. For example, nucleic acid constructs of the present invention can include sequences that hybridize to nucleic acid molecules that are fixed to chip, membrane, column, or microparticle, such that the nucleic acid molecules and/or translated complexes can be screened with one or more substances of interest.

Members of the libraries of the present invention may be labeled with a detectable label. The detectable label may be a radioisotope or a nonradioactive detectable molecule such as biotin, or other detectable moieties as they are known or developed in the art. The label may be directly or indirectly bound to the members of the library. Library members may be labeled by direct or indirect binding of a detectable marker to the nucleic acid or to a polypeptide of the library member.

Library with a Substance of Interest

A library of nucleic acid molecules can also include at least one substance of interest. The substance of interest can be bound with a nucleic acid molecule, a polypeptide, or complex of the present invention, can be unbound, or can be bound to some members of the library and not bound to other members of the library. The substance of interest can be directly bound or indirectly bound to a nucleic acid molecule of the present invention, but is preferably indirectly bound to a nucleic acid molecule of the present invention or directly bound to a complex of the present invention (particularly the polypeptide encoded by the random sequence or sequence of interest). Alternatively, the substance of interest can be a substrate, such as an enzymatic substrate, with which a nucleic acid molecule, polypeptide, or complex of the present invention interacts. Reactions of the nucleic acid molecule, polypeptide, or complex of the present invention with the substance of interest may be monitored and quantitated using appropriate assays, for example spectrophotometric assays or assays that measure the release of a radioactive moiety. One or more substances of interest can be directly or indirectly bound on a solid support (such as a chip or array, membrane, column, or microparticle) or can be in solution. Where more than one substance of interest is bound to a solid support for screening, the substances of interest can be provided on an addressable array or on tagged microparticles that can identify or locate a specific substance of interest in the plurality of substances of interest being screened.

In an alternative format, the structure formed by the construct of the present invention and the polypeptide encoded by a random sequence or sequence of interest can be immobilized on a solid support and one or more substances of interest may be unbound, may be bound with the fixed one or more complexes of the library, or may be acted upon in a biochemical reaction catalyzed by or modulated by one or more complexes of the library.

The substance of interest can be on or within a cell, wherein the cell can be ex vivo or in vivo, such as in a subject. The cell can be any cell, including a normal cell or an abnormal cell, such as, for example, a neoplastic cell or a virus infected cell. The substance of interest can also be one or a plurality of molecules on or within an abnormal or normal cell or on or within an etiological agent, such as, for example, a virus, a bacterium, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest may be a cell-type-specific or tissue-specific molecule, such that nucleic acids or peptides of the present invention that specifically bind to the substance of interest can be identified. Such cell-type-specific and tissue-specific molecules can be used to target drug delivery to specific cells or tissues.

The substance of interest can also include at least one organic molecule, an inorganic molecule, a polymer, a polypeptide, a lipid, a carbohydrate, a small molecule, a nucleic acid molecule, a ribozyme, a biomacromolecule or a drug.

Library of Vectors

The present invention also includes a library of vectors of the present invention. Preferably, the library of vectors includes at least two different random sequences, at least two different sequences of interest, or a combination or random sequences and sequences of interest.

### III Methods for Identifying Nucleic Acid Molecules

The present invention includes methods for identifying nucleic acid molecules, particularly from a library of random sequences or sequences of interest that encode polypeptides that bind with a substance of interest.

Methods Using Translation of Nucleic Acid Molecules of the Present Invention and Translation of Nucleic Acid Molecules Derived from Nucleic Acid Molecules of the Present Invention

This method includes: providing at least one nucleic acid molecule of the present invention as a single-stranded RNA, a double-stranded RNA, a single-stranded DNA, a double-stranded DNA, a single-stranded RNA/DNAchimera, a double-stranded RNA/DNA chimera, or a double-stranded RNA/DNA hybrid molecule that includes at least one random sequence or at least one sequence of interest; if the nucleic acid molecule is wholly or partially double-stranded, optionally converting the double stranded nucleic acid molecule or portions thereof to single stranded nucleic acid, translating the nucleic acid molecule to provide at least one complex, wherein the complex comprises a polypeptide operably linked to a random sequence or a nucleic acid sequence of interest or a nucleic acid molecule of interest; contacting at least one complex with at least one substance of interest; selecting at least one complex that binds with said at least one substance of interest; and identifying said random sequence or said nucleic acid sequence of interest or nucleic acid molecule of interest. The nucleic acid molecule, including the random sequence or selected sequence can be sequenced. Binding moiety/interacting domain complexes of the present invention, or a library thereof in the form of complexes, can be made by translating nucleic acid molecules of the present invention. Nucleic acid molecules of the present invention that are double-stranded, or that comprise double-stranded portions that are to be translated, are converted to single-stranded nucleic acid molecules prior to, or simultaneous with, translation. Methods of converting double-stranded nucleic acid molecules to single-stranded nucleic acid molecules are known in the art, and include the use of high temperature, changing the pH of the solution containing the DNA molecules, and the use of enzymes such as polymerases, exonucleases (Little, J. W. et al. (1967) J. Biol. Chem. 242, 672) or helicases. In addition, it may be desireable to use conditions that stabilize single-stranded forms of nucleic acids, such as low salt conditions or the presence of single-stranded nucleic acid binding proteins. Single-stranded templates can also be at least partially chemically or enzymatically synthesized.

Single-stranded RNA can be translated using methods known in the art, such as in vitro translation systems (Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Also, single-stranded DNA can be translated under certain conditions by ribosomes that use single-stranded DNA as a template and d(ATG) as a start codon (see, for example, Morgan et al. (1967) J Mol Biol 26: 477-497; Hulen et al. (1977) Biochimie 59:179-188; Salas and Bollum, J. (1969) Biol. Chem. 244:1152-1156; Bretscher (1968) Nature 220:1088-1091; Thorpe and Ihler (1974) Biochimica et Biophysica Acta 336:235-239; Ricker and Kaji (1991) Nucleic Acids Res. 19:6573-6578). The single-stranded DNA may be made from a variety of methods that are known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333).

The RNA portion of single-stranded RNA/DNA chimera can also be translated using methods known in the art (for example, Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Preferably, in a RNA/DNA chimera of the present invention, single-stranded DNA is 3′ of single-stranded RNA, but this is not required for the present invention. The DNA of said chimera is preferably linked to the RNA molecule using a method known to the art, such as by T4 DNA ligase. The DNA may also preferably be linked with a binding moiety. Alternatively, the DNA of the hybrid is double-stranded.

Following translation, it may be desirable to incubate the translation mixture under particular conditions of salt or temperature, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to complex free Mg²⁺ in the reaction mixture. This may be desirable for screening applications where the ribosome may impede binding to the substance of interest, impede the entry of complexes into cells, etc.

Complexes, including libraries of complexes, may be purified or substantially purified from the reaction mixture using reagents that bind parts of the complex. For example, if the spacer region encodes GST, the complexes may be purified by affinity chromatography using glutathione coupled to beads. Purified or substantially purified complexes may be stored under conditions that promote the stability of nucleic acids and polypeptides, for example, at 4° C. in a buffer that contains BSA and EDTA. Optionally, the single-stranded nucleic acid in said complex can be converted to double-stranded DNA using the methods known in the art, such as using reverse transcriptase or T4 DNA polymerase.

The complex is contacted with one or more substances of interest under conditions that promote the binding of the complex, or reaction of the complex, particularly the polypeptide encoded by the random sequence or sequence of interest, with the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method.

Complexes that are bound to the substance of interest via the nucleic acid of the complex may be eliminated from the selection procedure by any of the following methods. Prior to translation, the nucleic acid of the complex can be contacted with the substance of interest such that any nucleic acid with affinity for the substance of interest may bind with the substance of interest. The unbound nucleic acid is then recovered and used as the template in the transcription/translation or translation reaction. Alternatively, nonspecific RNA binding proteins such as hnRNPA1, La autoantigen, and the major core protein of cytoplasmid mRNP(p50) (Svitkin et al. (1996) EMBO J. 15: 7147-7155), double-stranded DNA binding proteins such as histones (Li, et al. Microbiol. 145: 1-2 (1999); Moenner et al., FEBS Lett. 443: 303-7 (1999); Grayling, et al., Extremophiles 1: 79-88 (1997)), and/or single-stranded DNA binding proteins (Ruvolo et al., Proteins 9: 120-34 (1991); Srivenugopal et al. Biochem. Biophys. Res. Commun. 137: 795-800 (1986)) may be added to the mixture of complexes after translation of the nucleic acids and before contacting the complexes with the substance of interest, such that the nucleic acid molecule is masked and thus unable to bind to the substance of interest. The nucleic acid binding proteins may be stripped from the sequence of interest prior to reverse transcription and/or amplification of the RNA or DNA of the complex using high temperature or denaturing agents such as phenol or guanidinium. Finally, removal of complexes that bind the substance of interest via the RNA component may be eliminated from selection after the first amplification of nucleic acids attached to bound complexes.

In this strategy, amplified nucleic acids of bound complexes are amplified and then cloned into a phage vector such that peptides encoded by the nucleic acids of the bound complexes are displayed on the phage surface. The phage are then selected by their binding to the substance of interest according to the “phage display” method (Smith and Petrenko (1997) Chem Rev 97:391-410; Spada and Pluckthun (1997) Nat Med 3:694-696). In this way only peptides encoded by the nucleic acids of the complexes, and not the nucleic acids themselves, are obtained after this second round of selection.

The selected complex or portions thereof, such as the polypeptide or the nucleic acid molecule encoding the polypeptide, can be isolated by recovery using a variety of methods. For example, changes in pH, detergents, denaturing agents (such as phenol, urea or quanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule. The nucleic acid molecule can be recovered using nucleic acid amplification procedures, such as PCR, using appropriate primers and methods.

The steps in this method can be performed reiteratively, such that recovered complexes or nucleic acid molecules can be contacted with the same or different substance of interest in order to increase the percentage of nucleic acid molecules that include a random sequence or a sequence of interest that encodes a polypeptide that binds with a substance of interest, such as for phage display or SELEX methods (see, for example, U.S. Pat. No. 5,747,253 to Ecker et al., issued May 5, 1998; U.S. Pat. No. 5,270,163 to Gold et al., issued Dec. 14, 1993; and Pasqualini and Ruoslahti, Nature 380:364-368 (1996)).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use the said method. These methods can be capillary affinity gel for nucleic acid and HPLC, or the combination thereof. The individual species of the nucleic acid molecules can then be sequenced. The amino acid sequence of the polypeptide that is able to bind to the substance of interest can be deduced from the nucleic acid sequence.

The entire selection procedure, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest.

Methods Using Transcription and Translation of Nucleic Acid Molecules of the Present Invention, Including Transcription and Translation of Nucleic Acid Molecules Derived from Nucleic Acid Molecules of the Present Invention

The present invention also includes a method for identifying a nucleic acid molecule or sequence that includes: providing at least one nucleic acid molecule of the present invention in the form of double-stranded DNA; transcribing the nucleic acid molecule to a corresponding RNA molecule; translating the RNA molecule to provide at least one complex, wherein the complex comprises a polypeptide operably linked to a random sequence or a nucleic acid sequence of interest or a nucleic acid molecule of interest; contacting the at least one complex with at least one substance of interest; selecting at least one complex that binds with the at least one substance of interest; and identifying said random sequence or nucleic acid sequence of interest or nucleic acid molecule of interest. Optionally, the nucleic acid molecule, including the random sequence or selected sequence, or the polypeptide corresponding thereto, can be sequenced or the polypeptide sequence can be deduced from nucleic acid sequences.

Any nucleic acid molecule of the present invention that can be converted to double-stranded DNA can be used in this embodiment of the present invention. For example, single-stranded DNA can be converted to double-stranded DNA using an appropriate nucleotide primer and a polymerase, such as the Klenow fragment of DNA polymerase I; single-stranded RNA may be converted to double-stranded DNA using an appropriate nucleotide primer, reverse transcriptase, and RNase H; double-stranded RNA may be converted to single-stranded RNA, and then converted to double-stranded DNA using an appropriate nucleotide primer, reverse transcriptase, and RNase H; RNA/DNA hybrids may be converted to double-stranded DNA by digesting the RNA strand of the hybrid with an enzyme such as RNase H, and then converting the single-stranded DNA to double-stranded DNA using an appropriate nucleotide primer and a polymerase, such as the Klenow fragment of DNA polymerase I. RNA/DNA chimeras that comprise double-stranded RNA, or RNA or DNA single-stranded portions may use one of the above methods to convert the double-stranded RNA, single-stranded RNA, or single-stranded DNA, portions of the chimera to double stranded DNA, as appropriate.

A nucleic acid molecule of the present invention or a library thereof in the form of complexes can be made by converting the double-stranded DNA molecule into an RNA molecule using methods known in the art, such as in vitro transcription systems and translation systems (for example, Pelham, H. R. B. (1976) Eur. J. Biochem. 67, 247; Zubay, G. (1973) Ann. Rev. Genet. 7, 267). The complex includes the random sequence or sequence of interest that encodes a polypeptide as well as that polypeptide itself. In one embodiment of the present invention (illustrated in FIG. 4), the nucleic acid construct of the present invention is in the form of double-stranded DNA, and in vitro transcription and translation of the construct are coupled, occurring simultaneously. Methods of performing linked transcription/translation in a single reaction are known in the art (see, for example, U.S. Pat. Nos. 5,324,637 and 5,492,817 to Thompson et al., and U.S. Pat. No. 5,665,563 to Beckler). In this embodiment, the construct preferably encodes a sequence or sequences encoding RNA sequences that are able to bind directly or indirectly to the interacting domain. Optionally, the construct also encodes a stem-forming sequence. Such stem-forming sequences are preferably at the 5′ end of the construct, such that upon transcription of the stem-forming sequences, a secondary structure forms at the 5′ end of the RNA that reduces the efficiency of translation of the RNA. Because translation occurs simultaneously with transcription, initial translation of the transcript may begin before stem-forming sequences are completely transcribed, and therefore before the stem structure has formed. In this way only one or a small number of translation events are able to occur before formation of the stem structure impedes translation. The interacting domain of the nascent polypeptide binds to the DNA to form a nucleic acid-polypeptide complex.

Transcription and translation reactions need not be linked or coupled, but can optionally be performed separately. Transcription can be performed using RNA polymerases known in the art, such as T7, T3, SP6, or E. coli RNA polymerases, or other polymerases that become known in the art, and can optionally use modified or non-naturally occurring ribonucleotides, such as, for example, phosphorothioate nucleotides, for synthesis of nuclease-resistant RNA molecules. Single-stranded RNA, including the single-stranded RNA portions of an RNA/DNA chimera can be translated using methods known in the art, such as in vitro translation systems (Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Translation systems can optionally include non-naturally occurring amino acyl tRNAs, such as amino acyl tRNAs charged with modified amino acids.

Following translation, it may be desirable to incubate the translation mixture under particular conditions of salt or temperature, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to complex free Mg²⁺ in the reaction mixture. This may be desirable for screening applications where the ribosome may impede binding to the substance of interest, or impede the entry of complexes into cells.

Complexes, including libraries of complexes, may be purified or substantially purified from the reaction mixture using reagents that bind parts of the complex. For example, if the spacer region encodes GST, the complexes may be purified by affinity chromatography using glutathione coupled to beads. Purified or substantially purified complexes may be stored under conditions that promote the stability of nucleic acids and polypeptides, for example, at 4° C. in a buffer that contains BSA and EDTA. Optionally, the RNA of the complex may be converted to an RNA/DNA duplex, or a DNA/DNA duplex using the methods known in the art, such as reverse transcription, primer extension and DNA ligation.

The complex is contacted with one or more substances of interest under conditions that promote the binding of the complex, particularly the polypeptide encoded by the random sequence or sequence of interest, to the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide region encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and one or more substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a ligand that binds with or reacts with a substance of interest has been selected using this method.

Complexes that are bound to the substance of interest via the nucleic acid of the complex may be eliminated from the selection procedure by any of the following methods. Prior to translation, the nucleic acid of the complex can be contacted with the substance of interest such that any nucleic acid with affinity for the substance of interest may bind with the substance of interest. The unbound nucleic acid is then recovered and used as the template in the transcription/translation or translation reaction. Alternatively, nonspecific RNA binding proteins such as hnRNPA1, La autoantigen, and the major core protein of cytoplasmid mRNP(p50) (Svitkin, et al. EMBO J. 15: 7147-7155(1996)), double-stranded DNA binding proteins such as histones (Li, et al. Microbiol. 145: 1-2 (1999); Moenner et al., FEBS Lett. 443: 303-7 (1999); Grayling, et al., Extremophiles 1: 79-88 (1997)), and/or single-stranded DNA binding proteins (Ruvolo et al., Proteins 9: 120-34 (1991); Srivenugopal et al. Biochem. Biophys. Res. Commun. 137: 795-800 (1986)) may be added to the mixture of complexes after translation of the nucleic acids and before contacting the complexes with the substance of interest, such that the nucleic acid molecule is masked and thus unable to bind to the substance of interest. The nucleic acid binding proteins may be stripped from the sequence of interest prior to reverse transcription and/or amplification of the RNA or DNA of the complex using high temperature or denaturing agents such as phenol or guanidinium. Finally, removal of complexes that bind the substance of interest via the RNA component may be eliminated from using multiple rounds of selection. Finally, amplified nucleic acids of bound complexes are amplified and then cloned into a phage vector such that peptides encoded by the nucleic acids of the bound complexes are displayed on the phage surface. The phage are then selected by their binding to the substance of interest according to the “phage display” method (Smith and Petrenko (1997) Chem Rev 97: 391-410; Spada and Pluckthun (1997) Nat Med 3: 694-696). In this way only peptides encoded by the nucleic acids of the complexes, and not the nucleic acids themselves, are obtained after this second round of selection.

The selected complex or portions thereof, such as the polypeptide or the nucleic acid molecule encoding the polypeptide, can be isolated by recovery using a variety of methods. For example, changes in pH, detergents, denaturing agents (such as phenol, urea or quanidinium), salt concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule. The nucleic acid molecule can be recovered using nucleic acid amplification procedures, such as PCR, using appropriate primers and methods.

The steps of this method can be performed reiteratively, such that recovered complexes or nucleic acid molecules can be contacted with the same or different substance of interest in order to increase the percentage of nucleic acid molecules that include a random sequence or a sequence of interest that encodes a polypeptide that binds with a substance of interest, such as for phage display or SELEX methods (see, for example, U.S. Pat. No. 5,747,253 to Ecker et al., issued May 5, 1998; U.S. Pat. No. 5,270,163 to Gold et al., issued Dec. 14, 1993; and Pasqualini and Ruoslahti, Nature 380:364-368 (1996)).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to convert the double-stranded PCR products to single-stranded form in order to use these methods. These methods can include, for example, capillary affinity gels for nucleic acids, HPLC, or combinations thereof. The individual species of the nucleic acid molecules can then be sequenced. The amino acid sequence of the polypeptide that is able to bind to the substance of interest can be deduced from the nucleic acid sequence.

The entire selection procedure, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that include at least a portion of a random sequence or selected nucleic acid sequence identified by this method. The present invention also includes polypeptides that include at least a portion of a polypeptide encoded by an identified random sequence or sequence of interest.

### IV Methods for Identifying Polypeptides

The present invention includes methods for identifying nucleic polypeptides, particularly polypeptides encoded by random sequences or sequences of interest that bind with a substance of interest.

Methods Using Translation of Nucleic Acid Molecules of the Present Invention and Translation of Nucleic Acid Molecules Derived from Nucleic Acid Molecules of the Present Invention

This aspect of the present invention includes a method for identifying a polypeptide encoded by a random nucleic acid sequence or nucleic acid sequence of interest or nucleic acid molecule of interest, including: providing at least one nucleic acid molecule of the present invention as a single-stranded RNA, a double-stranded RNA, a single-stranded DNA, a double-stranded DNA, a single-stranded RNA/DNA chimera, a double-stranded RNA/DNA chimera, or a double-stranded RNA/DNA hybrid molecule that includes at least one random sequence or at least one sequence of interest; if the nucleic acid molecule is wholly or partially double-stranded, optionally converting the double-stranded nucleic acid molecule or portions thereof to single-stranded nucleic acid, translating the nucleic acid molecule to provide at least one complex, wherein the complex comprises a polypeptide operably linked to a random sequence or a nucleic acid sequence of interest or a nucleic acid molecule of interest; contacting at least one complex with at least one substance of interest; selecting at least one complex that binds with said at least one substance of interest; and identifying said random sequence or said nucleic acid sequence of interest or nucleic acid molecule of interest. The nucleic acid molecule, including the random sequence or selected sequence can be sequenced. The corresponding polypeptide sequences can be deduced from the nucleic acid sequences.

Binding moiety/interacting domain complexes of the present invention, or a library thereof in the form of complexes, can be made by translating nucleic acid molecules of the present invention. Nucleic acid molecules of the present invention that are double-stranded, or that comprise double-stranded portions that are to be translated, are converted to single-stranded nucleic acid molecules prior to, or simultaneous with, translation. Methods of converting double-stranded nucleic acid molecules to single-stranded nucleic acid molecules are known in the art, and include the use of high temperature, changing the pH of the solution containing the DNA molecules, and the use of enzymes such as exonucleases (Little, J. W. et al. (1967) J. Biol. Chem. 242, 672) or helicases. In addition, it may be desirable to use conditions that stabilize single-stranded forms of nucleic acids, such as low salt conditions or the presence of single-stranded nucleic acid binding proteins.

Single-stranded RNA can be translated using methods known in the art, such as in vitro translation systems (Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Also, single-stranded DNA can be translated under certain conditions by ribosomes that use single-stranded DNA as a template and d(ATG) as a start codon (see, for example, Morgan et al. (1967) J Mol Biol 26: 477-497; Hulen et al. (1977) Biochimie 59:179-188; Salas and Bollum, J. (1969) Biol. Chem. 244:1152-1156; Bretscher (1968) Nature 220:1088-1091; Thorpe and Ihler (1974) Biochimica et Biophysica Acta 336:235-239; Ricker and Kaji (1991) Nucleic Acids Res. 19:6573-6578). The single-stranded DNA may be made from a variety of methods that are known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333).The RNA portion of single-stranded RNA/DNA chimera can also be translated using methods known in the art (for example, Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Preferably, in a RNA/DNA chimera of the present invention, single-stranded DNA is 3′ of single-stranded RNA, but this is not required for the present invention. The DNA of said chimera is preferably linked to the RNA molecule using a method known to the art, such as by T4 DNA ligase. The DNA may also preferably be linked with a binding moiety. Alternatively, the DNA of the hybrid is double-stranded.

Following translation, it may be desirable to incubate the translation mixture under particular conditions of salt or temperature, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to complex free Mg²⁺ in the reaction mixture. This may be desirable for screening applications where the ribosome may impede binding to the substance of interest, impede the entry of complexes into cells, eyc.

Complexes, including libraries of complexes, may be purified or substantially purified from the reaction mixture using reagents that bind parts of the complex. For example, if the spacer region encodes GST, the complexes may be purified by affinity chromatography using glutathione coupled to beads. Purified or substantially purified complexes may be stored under conditions that promote the stability of nucleic acids and polypeptides, for example, at 4° C. in a buffer that contains BSA and EDTA. Optionally, the single-stranded nucleic acid in said complex can be converted to double-stranded DNA using the methods known in the art, such as using reverse transcriptase or T4 DNA polymerase.

The complex is contacted with one or more substances of interest under conditions that promote the binding of the complex, or reaction of the complex, particularly the polypeptide encoded by the random sequence or sequence of interest, with the substance of interest. One or more substances of interest can be on a solid support or in solution. A solid support may be a bead or microparticle, including a labeled bead or microparticle. Beads can be labeled, for example, with chemical tags, flourophores, or radiofrequence tags. Methods of conjugating compounds to beads and microparticles are well known in the art. One or more substances of interest can also be provided on a solid support in the form of a chip or array. The substance of interest can be applied to the array surface by adsorption, chemical conjugation, photolithography, etc.

The substance of interest can be on or within a cell and can be on or within an etiological agent. Thus, a substance of interest is bound with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. Complexes of the present invention can be coupled to a solid support through their nucleic acid molecules, for example by hybridization to probes that are irreversibly bound to the solid support.

The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method. Complexes that are bound to the substance of interest via the nucleic acid of the complex may be eliminated from the selection procedure by any of the following methods. Prior to translation, the nucleic acid of the complex can be contacted with the substance of interest such that any nucleic acid with affinity for the substance of interest may bind with the substance of interest. The unbound nucleic acid is then recovered and used as the template in the transcription/translation or translation reaction. Alternatively, nonspecific RNA binding proteins such as hnRNPA1, La autoantigen, and the major core protein of cytoplasmid mRNP(p50) (Svitkin et al. (1996) EMBO J. 15: 7147-7155), double-stranded DNA binding proteins such as histones (Li, et al. Microbiol. 145: 1-2 (1999); Moenner et al., FEBS Lett. 443: 303-7 (1999); Grayling, et al., Extremophiles 1: 79-88 (1997)), and/or single-stranded DNA binding proteins (Ruvolo et al., Proteins 9: 120-34 (1991); Srivenugopal et al. Biochem. Biophys. Res. Commun. 137: 795-800 (1986)) may be added to the mixture of complexes after translation of the nucleic acids and before contacting the complexes with the substance of interest, such that the nucleic acid molecule is masked and thus unable to bind to the substance of interest. The nucleic acid binding proteins may be stripped from the sequence of interest prior to reverse transcription and/or amplification of the RNA or DNA of the complex using high temperature or denaturing agents such as phenol or guanidinium. Finally, removal of complexes that bind the substance of interest via the RNA component may be eliminated from selection after the first amplification of nucleic acids attached to bound complexes.

In this strategy, amplified nucleic acids of bound complexes are amplified and then cloned into a phage vector such that peptides encoded by the nucleic acids of the bound complexes are displayed on the phage surface. The phage are then selected by their binding to the substance of interest according to the “phage display” method (Smith and Petrenko (1997) Chem Rev 97:391-410; Spada and Pluckthun (1997) Nat Med 3:694-696). In this way only peptides encoded by the nucleic acids of the complexes, and not the nucleic acids themselves, are obtained after this second round of selection.

The selected complex or portions thereof, such as the polypeptide or the nucleic acid molecule encoding the polypeptide, can be isolated by recovery using a variety of methods. For example, changes in pH, detergents, denaturing agents (such as phenol, urea or quanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule. The nucleic acid molecule can be recovered using nucleic acid amplification procedures, such as PCR, using appropriate primers and methods.

The steps in this method can be performed reiteratively, such that recovered complexes or nucleic acid molecules can be contacted with the same or different substance of interest in order to increase the percentage of nucleic acid molecules that include a random sequence or a sequence of interest that encodes a polypeptide that binds with a substance of interest, such as for phage display or SELEX methods (see, for example, U.S. Pat. No. 5,747,253 to Ecker et al., issued May 5, 1998; U.S. Pat. No. 5,270,163 to Gold et al., issued Dec. 14, 1993; and Pasqualini and Ruoslahti, Nature 380:364-368 (1996)).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use the said method. These methods can be capillary affinity gel for nucleic acid and HPLC, or the combination thereof. The individual species of the nucleic acid molecules can then be sequenced. The amino acid sequence of the polypeptide that is able to bind to the substance of interest can be deduced from the nucleic acid sequence.

The entire selection procedure, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest. If the substance of interest is within a cell, the nucleic acid of the complex preferably encodes a peptide that mediates entry of associated molecules into the cell, for example, the translocating region of the tat protein of HIV (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999)). If the substance of interest is within a cell, the complex is preferably depleted of ribosomes prior to contacting the complex with the cells. A complex of the present invention can include a detectable label or a detectable domain such that the location of the nucleic acid molecule or complex of the present invention can be monitored on or in a cell.

Additionally, peptides may be identified that promote or inhibit cellular function using a variety of detection assays, such as, but not limited to, cellular assays. Assays may be transcription-based or may be based on a read-out that is not linked to transcription on a reporter gene, for example, the readout may depend on pH change, ion channel activity, changes in concentration of intracellular molecules such as Ca²⁺, or secretion by the cells of detectable molecules, etc. Cellular assays may require prescreening of the library to select for library members that interact with intracellular components of interest, and/or to screen out library members with undesirable binding properties.

Peptides encoded by random sequences or sequences of interest may also be selected for desirable catalytic functions. Assays may be developed in which novel, enhanced, or altered function of peptides of the present invention is detectable, for example colorometric assays or assays that measure the release of radioactive moieties from substrates.

Cellular and in vitro assays may be done in appropriate formats, such as in microtiter dishes and using plate readers. The complexes selected by such assays or portions thereof can be isolated using various purification methods and amplification methods as they are known in the art. For example, if the cellular or in vitro assay is performed in a microtiter format, complexes may be recovered from positively screening assay wells using antibodies or nucleic acids of complexes may be recovered from positively screening assay wells by amplification reactions using specific primers. Detergents, denaturing agents, and partial purification steps such as centrifugation may be used prior to recovery of the complexes or their components.

Where the substance of interest is on an etiological agent, such as a virus, a virus-infected cell, or a microbe, whole or lysed viruses or cells, or viral or cell extracts, may be used in the selection procedure. Binding may be performed with complexes or libraries of complexes to screen for peptides that bind to entities on or within viruses or cells, in order to identify peptides whose binding inhibits infectivity of the virus or etiological agent. In this application of the invention, multiple rounds of selection are performed. Complexes comprising peptides that bind the etiological agent are selected, and the nucleic acids of the complexes are amplified. The individual species of the PCR product can be sequenced and the polypeptide sequences can be deduced from the nucleic acid sequences. All or a portion of the individual sequenced polypeptides can be synthesized, preferably by solid phase synthesis. Then each peptide or portion thereof is added as to infectivity assays to determine whether the peptide inhibits infection by the etiological agent. The selected peptides that are able to inhibit infectivity of the etiological agent can also be used to identify a protein or proteins on said agent that mediate infectivity.

A preferred method for identifying such proteins is to construct a cDNA or genomic phage expression library of the agent, so that genes of the said agent are expressed on the surface of phages. The phage library is fixed on a nitrocellulose membrane as known in the art. The selected peptides can be used as the probes to select the phage clones that contain the genes corresponding to the proteins that binds with said selected peptide. The genes in the positive clones are sequenced and the genes of interest can thus be identified.

The recovered nucleic acid molecules that contain a random sequence or sequence of interest can be isolated using appropriate methods, such as gel electrophoresis. These sequences can be cloned into appropriate vectors, such as plasmids, which can be amplified in an appropriate host. The nucleic acid molecules can then be sequenced to determine the nucleic acid sequence of the random sequence or selected sequence. The amino acid sequence of the polypeptide encoded by the random sequence or sequence of interest can be determined directly by sequencing or by deducing the amino acid sequence that corresponds to the nucleic acid sequence of the random sequence or sequence of interest.

The present invention includes polypeptides identified by the present method, including polypeptides that include at least a portion of a polypeptide encoded by an identified random sequence or sequence of interest.

Peptides identified by their binding to cell-type-specific or tissue-specific molecules can be used to target delivery of therapeutic or toxic molecules to cells in vivo or ex vivo, including the cells of a patient. Drugs delivered by means of conjugation to or association with the peptides of the present invention may comprise nucleic acids, including gene therapy constructs, antisense constructs, and ribozymes; may comprise polypeptides; or may comprise organic or inorganic molecules, or may comprise a combination of any of these. Intracellular delivery of targeted drugs may be enhanced by the addition of peptides that promote the entry of attached molecules into cells, such as translocating domains of the tat and Antennapedia proteins (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999); Derossi, et al. J. Biol. Chem. 269: 10444-10450 (1994)), or other peptides or biomolecules as they are discovered in the art, including peptides and compounds discovered by the methods of the present invention. Furthermore, peptides that direct molecules to particular cellular compartments, for example, the endoplasmic reticulum or the mitochondria may also be linked to drugs for intracellular delivery.

Peptides identified by the methods of the present invention, such as peptides that may have therapeutic value, may themselves be linked or associated with peptides that target the peptides to particular cells or tissues and/or that mediate the entry of macromolecules into cells, such as the translocating domains of the tat and Antennapedia proteins. Furthermore, peptides that direct molecules to particular cellular compartments, for example, the endoplasmic reticulum or the mitochondria, or other intracellular localization sequences as they are known or may be identified in the art, may also be linked to peptides for intracellular delivery.

Peptides identified by the methods of the present invention may be directly or indirectly linked to a detectable label, such as a radioisotope, a small molecule such as biotin, a fluorescent protein such as green fluorescent protein, or an enzyme such as alkaline phospatase. Labels such as GFP and alkaline phosphatase can be encoded by constructs of the present invention. Labeled peptides of the present invention may be used in detection assays, including diagnostic assays. In addition, peptides of the present invention may be directly or indirectly linked to solid supports such as polymeric beads or nylon membranes, or other reagents used for purification of molecules, complexes, or cells. Peptides of the present invention may also be linked to therapeutic agents or toxins, such that the peptide of the present invention may increase the effectiveness of the therapeutic agent or toxin or by aiding the targeting of the therapeutic agent or toxin to particular tissues, microbes, cell types, or intracellular components.

Methods Using Transcription and Translation of Nucleic Acid Molecules of the Present Invention, Including Transcription and Translation of Nucleic Acid Molecules Derived from Nucleic Acid Molecules of the Present Invention

The present invention also includes a method for identifying a nucleic acid molecule or sequence that includes: providing at least one nucleic acid molecule of the present invention in the form of single-stranded DNA or double-stranded DNA; converting the nucleic acid molecule to a corresponding RNA molecule; translating the RNA molecule to provide at least one complex, wherein the complex comprises a polypeptide operably linked to a random sequence or a nucleic acid sequence of interest or a nucleic acid molecule of interest; contacting the at least one complex with at least one substance of interest; selecting at least one complex that binds with the at least one substance of interest; and identifying the polypeptide in said complex. Optionally, the nucleic acid molecule, including the random sequence or selected sequence, or the polypeptide corresponding thereto, can be sequenced or the sequence deduced from nucleic acid sequences.

Any nucleic acid molecule of the present invention that can be converted to double-stranded DNA can be used in this embodiment of the present invention. For example, single-stranded DNA can be converted to double-stranded DNA using an appropriate nucleotide primer and a polymerase, such as the Klenow fragment of DNA polymerase I; single-stranded RNA may be converted to double-stranded DNA using an appropriate nucleotide primer, reverse transcriptase, and RNase H; double stranded RNA may be converted to single-stranded RNA, and then converted to double-stranded DNA using an appropriate nucleotide primer, reverse transcriptase, and RNase H; RNA/DNA hybrids may be converted to double-stranded DNA by digesting the RNA strand of the hybrid with an enzyme such as RNase H, and then converting the single-stranded DNA to double-stranded DNA using an appropriate nucleotide primer and a polymerase, such as the Klenow fragment of DNA polymerase I. RNA/DNA chimeras that comprise double-stranded RNA, or single-stranded RNA or DNA portions may use one of the above methods to convert the double-stranded RNA, single-stranded RNA, or single-stranded DNA, portions of the chimera to double-stranded DNA.

A nucleic acid molecule of the present invention or a library thereof in the form of complexes can be made by converting the double-stranded DNA molecule into an RNA molecule and a complex of the present invention using methods known in the art, such as in vitro transcription systems and translation systems (for example, Anderson, C. W. et al. (1983) Meth. Enzymol. 101, 635; Pelham, H. R. B. (1976) Eur. J. Biochem. 67, 247; Zubay, G. (1973) Ann. Rev. Genet. 7, 267). The complex includes the random sequence or sequence of interest that encodes a polypeptide as well as that polypeptide itself. In one embodiment of the present invention (illustrated in FIG. 4), the nucleic acid construct of the present invention is in the form of double-stranded DNA, and in vitro transcription and translation of the construct are coupled, occurring simultaneously. Methods of performing linked transcription/translation in a single reaction are known in the art (see, for example, U.S. Pat. Nos. 5,324,637 and 5,492,817 to Thompson et al., and U.S. Pat. No. 5,665,563 to Beckler). In this embodiment, the construct preferably encodes a sequence or sequences encoding RNA sequences that are able to bind directly or indirectly to the interacting domain. Optionally, the construct also encodes a stem-forming sequence. Such stem-forming sequences are preferably at the 5′ end of the construct, such that upon transcription of the stem-forming sequences, a secondary structure forms at the 5′ end of the RNA that reduces the efficiency of translation of the RNA. Because translation occurs simultaneously with transcription, initial translation of the transcript may begin before stem-forming sequences are completely transcribed, and therefore before the stem structure has formed. In this way only one or a small number of translation events are able to occur before formation of the stem structure impedes translation. The interacting domain of the nascent polypeptide binds to the DNA to form a nucleic acid-polypeptide complex.

Transcription and translation reactions need not be linked or coupled, but can optionally be performed separately. Transcription can be performed using RNA polymerases known in the art, such as T7, T3, SP6, or E. coli RNA polymerases, or other polymerases that become known in the art, and can optionally use modified or non-naturally occurring ribonucleotides, such as, for example, phosphorothioate nucleotides, for synthesis of nuclease-resistant RNA molecules. Single-stranded RNA, including the single-stranded RNA portions of an RNA/DNA chimera can be translated using methods known in the art, such as in vitro translation systems (Anderson, C. W. et al. (1983) Meth. Enzymol. 101, 635; Pelham, H. R. B. and Jackson, R. J. (1976) Eur. J. Biochem. 67, 247; Zubay, G. (1973), Ann. Rev. Genet. 7, 267). Translation systems can optionally include non-naturally occurring amino acyl tRNAs, such as amino acyl tRNAs charged with modified amino acids.

Following translation, it may be desirable to incubate the translation mixture under particular conditions of salt or temperature, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to complex free Mg²⁺ in the reaction mixture. This may be desirable for screening applications where the ribosome may impede binding to the substance of interest, or impede the entry of complexes into cells.

Complexes, including libraries of complexes, may be purified or substantially purified from the reaction mixture using reagents that bind parts of the complex. For example, if the spacer region encodes GST, the complexes may be purified by affinity chromatography using glutathione coupled to beads. Purified or substantially purified complexes may be stored under conditions that promote the stability of nucleic acids and polypeptides, for example, at 4° C. in a buffer that contains BSA and EDTA.

Optionally, the RNA of the complex may be converted to RNA/DNA duplex, or DNA/DNA duplex using the methods known in the art, such as reverse transcription, primer extension and DNA ligation.

The complex is contacted with one or more substances of interest under conditions that promote the binding of the complex, particularly the polypeptide encoded by the random sequence or sequence of interest, to the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide region encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and one or more substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a ligand that binds with or reacts with a substance of interest has been selected using this method.

Complexes that are bound to the substance of interest via the nucleic acid of the complex may be eliminated from the selection procedure by any of the following methods. Prior to translation, the nucleic acid of the complex can be contacted with the substance of interest such that any nucleic acid with affinity for the substance of interest may bind with the substance of interest. The unbound nucleic acid is then recovered and used as the template in the transcription/translation or translation reaction. Alternatively, nonspecific RNA binding proteins such as hnRNPA1, La autoantigen, and the major core protein of cytoplasmid mRNP(p50) (Svitkin, et al. EMBO J. 15: 7147-7155(1996)), double-stranded DNA binding proteins such as histones (Li, et al. Microbiol. 145: 1-2 (1999); Moenner et al., FEBS Lett. 443: 303-7 (1999); Grayling, et al., Extremophiles 1: 79-88 (1997)), and/or single-stranded DNA binding proteins (Ruvolo et al., Proteins 9: 120-34 (1991); Srivenugopal et al. Biochem. Biophys. Res. Commun. 137: 795-800 (1986)) may be added to the mixture of complexes after translation of the nucleic acids and before contacting the complexes with the substance of interest, such that the nucleic acid molecule is masked and thus unable to bind to the substance of interest. The nucleic acid binding proteins may be stripped from the sequence of interest prior to reverse transcription and/or amplification of the RNA or DNA of the complex using high temperature or denaturing agents such as phenol or guanidinium. Finally, removal of complexes that bind the substance of interest via the RNA component may be eliminated from using multiple rounds of selection. In this strategy, amplified nucleic acids of bound complexes are amplified and then cloned into a phage vector such that peptides encoded by the nucleic acids of the bound complexes are displayed on the phage surface. The phage are then selected by their binding to the substance of interest according to the “phage display” method (Smith and Petrenko (1997) Chem Rev 97: 391-410; Spada and Pluckthun (1997) Nat Med 3: 694-696). In this way only peptides encoded by the nucleic acids of the complexes, and not the nucleic acids themselves, are obtained after this second round of selection.

The selected complex or portions thereof, such as the polypeptide or the nucleic acid molecule encoding the polypeptide, can be isolated by recovery using a variety of methods. For example, changes in pH, detergents, denaturing agents (such as phenol, urea or quanidinium), salt concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule. The nucleic acid molecule can be recovered using nucleic acid amplification procedures, such as PCR, using appropriate primers and methods.

The steps of this method can be performed reiteratively, such that recovered complexes or nucleic acid molecules can be contacted with the same or different substance of interest in order to increase the percentage of nucleic acid molecules that include a random sequence or a sequence of interest that encodes a polypeptide that binds with a substance of interest, such as for phage display or SELEX methods (see, for example, U.S. Pat. No. 5,747,253 to Ecker et al., issued May 5, 1998; U.S. Pat. No. 5,270,163 to Gold et al., issued Dec. 14, 1993; and Pasqualini and Ruoslahti, Nature 380:364-368 (1996)).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to convert the double-stranded PCR products to single-stranded form in order to use these methods. These methods can include, for example, capillary affinity gel for nucleic acids, HPLC, or the combination of thereof. The individual species of the nucleic acid molecules can then be sequenced. The amino acid sequence of the polypeptide that is able to bind to the substance of interest can be deduced from the nucleic acid sequence.

The entire selection procedure, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that include at least a portion of a random sequence or selected nucleic acid sequence identified by this method. The present invention also includes polypeptides that include at least a portion of a polypeptide encoded by an identified random sequence or sequence of interest. If the substance of interest is within a cell, the nucleic acid of the complex preferably encodes a peptide that mediates entry of associated molecules into the cell, for example, the translocating region of the tat protein of HIV (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999)). If the substance of interest is within a cell, the complex is preferably depleted of ribosomes prior to contacting the complex with the cells. A complex of the present invention can include a detectable label or a detectable domain such that the location of the nucleic acid molecule or complex of the present invention can be monitored on or in a cell.

Additionally, peptides may be identified that promote or inhibit cellular function using a variety of detection assays, such as, but not limited to, cellular assays. Assays may be transcription-based or may be based on a read-out that is not linked to transcription on a reporter gene, for example, the readout may depend on pH change, ion channel activity, changes in concentration of intracellular molecules such as Ca²⁺, or secretion by the cells of detectable molecules, etc. Cellular assays may require prescreening of the library to select for library members that interact with intracellular components of interest, and/or to screen out library members with undesirable binding properties.

Peptides encoded by random sequences or sequences of interest may also be selected for desirable catalytic functions. Assays may be developed in which novel, enhanced, or altered function of peptides of the present invention is detectable, for example colorometric assays or assays that measure the release of radioactive moieties from substrates.

Cellular and in vitro assays may be done in appropriate formats, such as in microtiter dishes and using plate readers. The complexes selected by such assays or portions thereof can be isolated using various purification methods and amplification methods as they are known in the art. For example, if the cellular or in vitro assay is performed in a microtiter format, complexes may be recovered from positively screening assay wells using antibodies or nucleic acids of complexes may be recovered from positively screening assay wells by amplification reactions using specific primers. Detergents, denaturing agents, and partial purification steps such as centrifugation may be used prior to recovery of the complexes or their components.

Where the substance of interest is on an etiological agent, such as a virus, a virus-infected cell, or a microbe, whole or lysed viruses or cells, or viral or cell extracts, may be used in the selection procedure. Binding may be performed with complexes or libraries of complexes to screen for peptides that bind to entities on or within viruses or cells, in order to identify peptides whose binding inhibits infectivity of the virus or etiological agent. In this application of the invention, multiple rounds of selection are performed. Complexes comprising peptides that bind the etiological agent are selected, and the nucleic acids of the complexes are amplified. The individual species of the PCR product can be sequenced and the polypeptide sequences can be deduced from the nucleic acid sequences. All or a portion of the individual sequenced polypeptides can be synthesized, preferably by solid phase synthesis. Then each peptide or portion thereof is added as to infectivity assays to determine whether the peptide inhibits infection by the etiological agent. The selected peptides that are able to inhibit infectivity of the etiological agent can also be used to identify a protein or proteins on said agent that mediate infectivity.

A preferred method for identifying such proteins is to construct a cDNA or genomic phage expression library of the agent, so that genes of the said agent are expressed on the surface of phages. The phage library is fixed on a nitrocellulose membrane as known in the art. The selected peptides can be used as the probes to select the phage clones that contain the genes corresponding to the proteins that binds with said selected peptide. The genes in the positive clones are sequenced and the genes of interest can thus be identified.

The recovered nucleic acid molecules that contain a random sequence or sequence of interest can be isolated using appropriate methods, such as gel electrophoresis. These sequences can be cloned into appropriate vectors, such as plasmids, which can be amplified in an appropriate host. The nucleic acid molecules can then be sequenced to determine the nucleic acid sequence of the random sequence or selected sequence. The amino acid sequence of the polypeptide encoded by the random sequence or sequence of interest can be determined directly by sequencing or by deducing the amino acid sequence that corresponds to the nucleic acid sequence of the random sequence or sequence of interest.

The present invention includes polypeptides identified by the present method, including polypeptides that include at least a portion of a polypeptide encoded by an identified random sequence or sequence of interest.

Peptides identified by their binding to cell-type-specific or tissue-specific molecules can be used to target delivery of therapeutic or toxic molecules to cells, such as the cells of a patient. Drugs delivered by means of conjugation to or association with the peptides of the present invention may comprise nucleic acids, including gene therapy constructs, antisense constructs, and ribozymes; may comprise polypeptides; or may comprise organic or inorganic molecules, or may comprise a combination of any of these. Intracellular delivery of targeted drugs may be enhanced by the addition of peptides that promote the entry of attached molecules into cells, such as translocating domains of the tat and Antennapedia proteins (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999); Derossi, et al. J. Biol. Chem. 269: 10444-10450 (1994)), or other peptides or biomolecules as they are discovered in the art, including peptides and compounds discovered by the methods of the present invention. Furthermore, peptides that direct molecules to particular cellular compartments, for example, the endoplasmic reticulum or the mitochondria may also be linked to drugs for intracellular delivery.

Peptides identified by the methods of the present invention, such as peptides that may have therapeutic value, may themselves be linked or associated with peptides that target the peptides to particular cells or tissues and/or that mediate the entry of macromolecules into cells, such as the translocating domains of the tat and Antennapedia proteins. Furthermore, peptides that direct molecules to particular cellular compartments, for example, the endoplasmic reticulum or the mitochondria may also be linked to peptides for intracellular delivery.

Peptides identified by the methods of the present invention may be directly or indirectly linked to a detectable label, such as a radioisotope, a small molecule such as biotin, a fluorescent protein such as green fluorescent protein (GFP), or an enzyme such as alkaline phospatase. Labels such as GFP and alkaline phosphatase can be encoded by a construct of the present invention. Labeled peptides of the present invention may be used in detection assays, including diagnostic assays. In addition, peptides of the present invention may be directly or indirectly linked to solid supports such as polymeric beads or nylon membranes, or other reagents used for purification of molecules, complexes, or cells. Peptides of the present invention may also be linked to therapeutic agents or toxins, such that the peptide of the present invention may increase the effectiveness of the therapeutic agent or toxin or by aiding the targeting of the therapeutic agent or toxin to particular tissues, microbes, cell types, or intracellular components.

### V Methods for Identifying Test Compounds

The present invention includes methods for identifying test compounds, test compounds identified by this method and pharmaceutical compositions identified by this method.

One aspect of the present invention is a method for identifying a test compound, including: contacting a target with a complex that comprises a nucleotide sequence that: comprises a binding moiety, encodes an interacting domain, and comprises a random sequence or a sequence of interest that encodes a polypeptide, wherein the interacting domain directly or indirectly binds with the binding moiety; identifying complexes bound with said target, or on the basis of catalytic function or the results of cellular assays; determining the structure of the polypeptide encoded by the random sequence or sequence of interest; and identifying moieties for use as test compounds that have structures that have space filling shapes that are similar to at least a portion of said identified moiety. The present invention also includes a test compound identified by this method and a pharmaceutical composition identified by this method.

Complexes, nucleic acid molecules and polypeptides of the present invention that bind with a substance of interest, such as a target, including a pharmaceutical target, or complexes that comprise peptides or nucleic acids with desirable catalytic properties, can be identified using methods of the present invention. The structure of the identified nucleic acid molecule or amino acid can be determined using methods such as, for example, NMR and mass spectroscopy. Alternatively, the identified nucleic acid molecule sequences or amino acid sequences can be provided to a processing unit and appropriate computer models and software to model the three dimensional configuration of the peptide that binds the target encoded therein. Appropriate computer models and software can also provide structures of chemical libraries that correspond to or are related to at least a portion of the three dimensional configuration. These chemical libraries can be synthesized in whole or in part by combinatorial chemistry methodologies. These libraries can then be screened for activity, such as pharmacological activity, using methods known in the art and described herein. Alternatively or in addition, peptides identified by the methods of the present invention can be modified or derivatized and the modified or derivatized forms can be screened for optimized properties and functions of the peptide.

Pharmacology and Toxicity of Test Compounds

The structure of a test compound can be determined or confirmed by methods known in the art, such as mass spectroscopy. For test compounds stored for extended periods of time under a variety of conditions, the structure, activity and potency thereof can be confirmed.

Identified test compounds can be evaluated for a particular activity using are-recognized methods and those disclosed herein. For example, if an identified test compound is found to have anticancer cell activity in vitro, then the test compound would have presumptive pharmacological properties as a chemotherapeutic to treat cancer. Such nexuses are known in the art for several disease states, and more are expected to be discovered over time. Based on such nexuses, appropriate confirmatory in vitro and in vivo tests of pharmacological activity, and toxicology, and be selected and performed. The methods described herein can also be used to assess pharmacological selectivity and specificity, and toxicity.

Identified test compounds can be evaluated for toxicological effects using known methods (see, Lu, Basic Toxicology, Fundamentals, Target Organs, and Risk Assessment, Hemisphere Publishing Corp., Washington (1985); U.S. Pat. No; 5,196,313 to Culbreth (issued Mar. 23, 1993) and U.S. Pat. No. 5,567,952 to Benet (issued Oct. 22, 1996)). For example, toxicology of a test compound can be established by determining in vitro toxicity towards a cell line, such as a mammalian, for example human, cell line. Test compounds can be treated with, for example, tissue extracts, such as preparations of liver, such as microsomal preparations, to determine increased or decreased toxicological properties of the test compound after being metabolized by a whole organism. The results of these types of studies are predictive of toxicological properties of a chemical in animals, such as mammals, including humans.

Alternatively, or in addition to these in vitro studies, the toxicological properties of a test compound in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the test compound, the skilled artisan would not be burdened to determine appropriate doses, LD₅₀ values, routes of administration and regimes that would be appropriate to determine the toxicological properties of the test compound. In addition to animal models, human clinical trials can be performed following established procedures, such as those set forth by the United States Food and Drug Administration (USFDA) or equivalents of other governments. These toxicity studies provide the basis for determining the efficacy of a test compound in vivo.

Efficacy of Test Compounds

Efficacy of a test compound can be established using several art recognized methods, such as in vitro methods, animal models or human clinical trials (see, Creasey, supra (1979)). Recognized in vitro models exist for several diseases or conditions. For example, the ability of a test compound to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic, disease or condition, an acceptable in vitro or animal model is available. The skilled artisan is armed with a wide variety of such models as they are available in the literature or from the USFDA or the National Institutes of Health (NIH). In addition, these in vitro methods can use tissue extracts, such as preparations of liver, such as microsomal preparations, to provide a reliable indication of the effects of metabolism on a test compound. Similarly, acceptable animal models can be used to establish efficacy of test compounds to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model which confirms the validity of this model (see, McDonough, Phys. Ther. 62:835-839 (1982)). When choosing an appropriate model to determine efficacy of test compounds, the skilled artisan can be guided by the state of the art, the USFDA or the NIH to choose an appropriate model, doses and route of administration, regime and endpoint and as such would not be unduly burdened. In addition to animal models, human clinical trials can be used to determine the efficacy of test compounds. The USFDA, or equivalent governmental agencies, have established procedures for such studies.

Selectivity of Test Compounds

The in vitro and in vivo methods described above also establish the selectivity of a candidate modulator. It is recognized that chemicals can modulate a wide variety of biological processes or be selective. Panels of cells as they are known in the art can be used to determine the specificity of the a test compound (WO 98/13353 to Whitney et al., published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a test compound can be established in vitro by testing the toxicity and effect of a test compound on a plurality of cell lines that exhibit a variety of cellular pathways and sensitivities. The data obtained form these in vitro toxicity studies can be extended to animal model studies, including human clinical trials, to determine toxicity, efficacy and selectivity of a test compound.

The selectivity, specificity and toxicology, as well as the general pharmacology, of a test compound can be often improved by generating additional test compounds based on the structure/property relationship of a test compound originally identified as having activity. Test compounds can be modified to improve various properties, such as affinity, life-time in blood, toxicology, specificity and membrane permeability. Such refined test compounds can be subjected to additional assays as they are known in the art or described herein. Methods for generating and analyzing such compounds or compositions are known in the art, such as U.S. Pat. No. 5,574,656 to Agrafiotis et al.

### PHARMACEUTICAL COMPOSITIONS

The present invention also encompasses a test compound in a pharmaceutical composition comprising a pharmaceutically acceptable carrier prepared for storage and preferably subsequent administration, which have a pharmaceutically effective amount of the test compound in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The test compounds of the present invention can be formulated and used as tablets, capsules or elixirs for oral administration; suppositories for rectal administration; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as liposomes, can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperiotoneally, employing a variety of dosage forms. Such methods can also be used in testing the activity of test compounds in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the test compounds.

In non-human animal studies, applications of the pharmaceutical compositions are commenced at higher dose levels, with the dosage being decreased until the desired effect is no longer achieved or adverse side effects are reduced of disappear. The dosage for the test compounds of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, nasal, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated. Agents intended to be administered intracellularly may be administered using techniques well known to those of ordinary skill in the art. For example, such agents may be encapsulated into liposomes, then administered as described above. Intracellular delivery of drugs may be acheived by linking peptides such as the translocating domain of the tat protein of HIV to the agent. Linkage of hydrophobic molecules such as biotin to the attached tat peptide or similar translocating peptides may improve intracellular delivery further (Chen et al. Analyt. Biochem. 227: 168-175 (1995)). Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions. Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The test compounds of the present invention, and pharmaceutical compositions that include such test compounds are useful for treating a variety of ailments in a patient, including a human. As set forth in the Examples, the test compounds of the present invention have antibacterial, antimicrobial, antiviral, anticancer cell, antitumor and cytotoxic activity. A patient in need of such treatment can be provided a test compound of the present invention, preferably in a pharmacological composition in an effective amount to reduce the number or growth rate of bacteria, microbes, cancer cells or tumor cells in said patent, or to reduce the infectivity of viruses in said patient. The amount, dosage, route of administration, regime and endpoint can all be determined using the procedures described herein or by appropriate government agencies, such as the United Stated Food and Drug Administration.

### VI Methods for Identifying Targets

The present invention includes methods for identifying targets such as pharmaceutical targets, purification targets or diagnostic targets. The present invention also includes targets and pharmaceutical targets identified by such methods.

Another aspect of the present invention are methods for identifying a target, such as a pharmaceutical target, that include: contacting a substance of interest with a complex that: comprises a binding moiety, encodes an interacting domain, and comprises a random sequence or a sequence of interest, wherein the interacting domain directly or indirectly binds with the binding moiety; identifying targets that bind with the complex. The present invention also includes a target identified by this method, including pharmaceutical targets.

In one application of the invention, complexes comprising peptides that bind an etiological agent, either on the surface of the etiological agent, or to internal components of the etiological agent are selected, and the nucleic acids of the complexes are amplified as described in previous sections. The individual species of the PCR product can be sequenced and the polypeptide sequences are deduced from the nucleic acid sequences. Polypeptides can be synthesized using the deduced sequences, preferably by solid phase synthesis, although other methods of synthesis, such as expression of the encoding nucleic acids in vivo or in vitro, may also be used. Then each synthesized peptide is added to infectivity assays to determine whether the peptide inhibits infection by the etiological agent. Any of the selected peptides that are able to inhibit infectivity of the etiological agent can also be used as probes to screen a library, such as a phage cDNA expression library, derived from the etiological agent. Library members that are selected by the probes may contain one or more genes or parts of genes that are responsible for the infectivity of the etiological agent. The proteins encoded by the genes identified in this way are thus identified as drug targets.

If the potential target is within a cell, the nucleic acid of the complex preferably encodes a peptide that mediates entry of associated molecules into the cell, for example, the translocating region of the tat protein of HIV. If the potential target is within a cell, the complex is preferably depleted of ribosomes (for example, by complexing free Mg²⁺ with EDTA) prior to contacting the complex with the cells. A complex of the present invention can include a detectable label or a detectable domain such that the location of the nucleic acid molecule of complex of the present invention can be monitored on or in a cell. Additionally, peptides may be identified that promote or inhibit cellular functions using a variety of detection assays. Such identified peptides can be used for identification and isolation of cellular targets.

Assays may be transcription-based or may be based on a read-out that is not linked to transcription on a reporter gene, for example, the readout may depend on pH change, ion channel acrtivity, changes in concentration of intracellular molecules such as Ca²⁺, or secretion by the cells of detectable molecules, etc. Cellular assays may require prescreening of the library to select for library members that interact with intracellular components of interest, and/or to screen out library members with undesireable binding properties.

In addition, the ability of a complex to modulate signal transduction pathways can be determined. The ability of a complex to modulate an identified signal transduction pathways can identify such signal transduction pathway as a therapeutic target. A variety of cells that comprise reporter genes that report an increased or decreased activity of a signal transduction pathway in response to a compound are known in the art. Such cells can also be made using methods known in the art (see, WO 98/13353 to Whitney, published Apr. 2, 1999; U.S. Pat. No. 5,298,429 to Evans et al., issued Mar. 29, 1994; and Skarnes et al., Genes and Development 6:903-918 (1992)). Complexes of the present invention can be contacted with such cells and the expression of the reporter gene monitored to identify signal transduction pathways modulated by the complex. Such identified signal transduction pathways can themselves be pharmaceutical targets, including the individual components of the identified signal transduction pathway Peptides encoded by random sequences or sequences of interest may also be selected for desireable catalytic functions. Assays may be developed in which enhanced or altered function of peptides of the present invention is detectable, for example colorometric assays or assays that measure the release of radioactive moieties from substrates.

Cellular and in vitro assays may be done in appropriate formats, such as in microtiter dishes and using plate readers. The complexes selected by such assays or portions thereof can be isolated using various purification methods and amplification methods as they are known in the art. For example, if the cellular or in vitro assay is performed in a microtiter format, complexes may be recovered from positively screening assay wells using antibodies or nucleic acids of complexes may be recovered from positively screening assay wells by amplification reactions using specific primers. Detergents, denaturing agents, and partial purification steps such as centrifugation may be used prior to recovery of the complexes or their components.

### EXAMPLES

### Example I: Making a Construct for Generating a Random Sequence Library

This example provides methods for making nucleic acid constructs that are useful for making one or more constructs that can include a random sequence that can be used in a method of the present invention.

Introducting bFGF Gene into Plasmid pGEX-5X-1

The 18kDa bFGF cDNA (Genbank Accession No. M27968) is obtained by using PCR that employs a pair of primers, pFG-aug and pFG-end, such as SEQ ID NO:1 and SEQ ID NO:2, a human brain cDNA library (Clontech, CA) as the template, and High Fidelity Klen Taq DNA polymerase (Clontech, CA) to amplify the open reading frame (ORF) of the 18 kDa bFGF according to the manufacturer's protocol (see also Innis et al. (eds.), PCR protocols: A Guide to Methods and Applications, Academic Press, San Diego, Calif. (1989)). The PCR product is approximately 500 bp in size. The DNA is purified by phenol/chloroform extraction and isopropanol precipitation. The precipitated DNA is dissolved in deionized water. The DNA is then digested by HindIII and XbaI in the restriction buffer 2 (New England Biolabs, MA) at 37 C. for 1 hour. The reaction is resolved on a 1% agarose gel and the DNA at approx. 500 bp is purified from the gel using Nucleospin gel purification columns (Clontech, CA). The DNA sequence is confirmed by sequencing or restriction enzyme digestion. Accordingly, a DNA fragment is generated that contains the 18 kDa human bFGF cDNA having a HindIII sticky-end at the 5′ of the gene and a XbaI sticky-end at the 3′ of the gene.

Plasmid pGEX-5X-1, a bacterial expression vector containing the gene for glutathione-S-transferase (GST) is commercially available (Pharmacia, NJ). The plasmid is linearized by EcoNI. The linearized plasmid DNA is amplified by PCR employing a pair of primers, GEX-F285 and GEX-R239 such as SEQ ID NO:3 and SEQ ID NO:4), that contain restriction enzyme recognition sites for Xba I and HindIII, respectively. DyNAzyme EXT DNA polymerase (MJ Research, MA), a polymerase that is capable of amplifying long DNA templates while maintaining high sequence fidelity is used in the amplification reaction. The PCR product is purified using standard phenol/chloroform extraction and isopropanol precipitation. The precipitated DNA is dissolved in deionized water and digested by HindIII and XbaI in restriction buffer 2 (New England Biolabs, MA) at 37 C. for 1 hour. The reaction is then resolved in a 1% agarose gel and the corresponding DNA at approx. 5 kb is purified using the Nucleospin (TM) gel extraction kit (Clontech, CA). The gel-purified DNA is the linearized modified pGEX-5X-1 vector that bears the XbaI sticky-end at the 5′ of the GST gene and the HindIII sticky-end at other end of the linearized plasmid.

The purified bFGF cDNA fragment and the linearized modified pGEX-5X-1 are ligated by T4-ligase (New England Biolabs, MA) to generate the plasmid pFG. The ligated DNA is used to transform competent E. coli DH5 alpha. The transformant colonies are picked and expanded. The plasmid pFG is purified and the correct orientation of the insert is confirmed using BamHI restriction enzyme digestion of the bFGF-GST gene. The junction sequence of the bFGF and GST gene is sequenced to confirm the correct reading frame.

Introducing T7 RNA Polymerase Promoter and MBR (Binding Moiety) Sequence into the 5′-untranslated Region (5′-UTR) of the bFGF-GST Gene

The plasmid pFG is linearized using KpnI and NcoI restriction enzymes in restriction buffer 1 (New England Biolabs, Beverly, Mass.). The linearized plasmid is resolved in a 1% agarose gel and purified using the Nucleospin gel extraction kit (Clontech, CA). Two oligonucleotides, T7-MBR1 and T7-MBR2, such as SEQ ID NO:5 and SEQ ID NO:6, are synthesized (Biogenosys, TX). The two oligonucleotides have a stretch of 1 6-nucleotide complementary sequence to each other at their 3′-ends. The two oligonucleotides are annealed by the 3′-end complementary sequences and then each oligonucleotide is extended outward from the double-stranded region using the opposite oligonucleotide as a template using T4 DNA polymerase (New England Biolabs, MA) at about 37 C. for about 2 hours. In this way a double-stranded (ds-) DNA, called T7-MBR (MBR, binding moiety) is synthesized. The T7-MBR contains a promoter sequence for T7 RNA polymerase and a 34-nucleotide region (MBR) encoding a RNA that binds to the 18 kDa bFGF (Jellinek et al., Proc. Natl. Acad. Sci. USA 90: 11227-31 (1993)). The T7-MBR is digested by KpnI and NcoI in restriction buffer 1 (New England Biolabs, Beverly, Mass.) to create a sticky-end at each end and purified using the Nucleotrap (TM) gel extraction kit (Clontech, CA).

The linearized pFG and T7-MBR are ligated using T4 DNA ligase (New England Biolabs, Beverly, Mass.) to generate plasmid pUFG. The ligation reaction is used to transform competent E. coli DH5alpha. The transformant colonies are picked and expanded, and the plasmid pUFG is recovered.

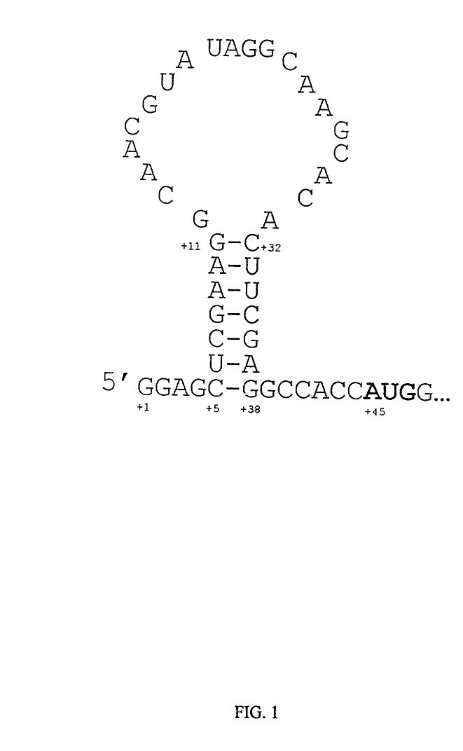

The pUFG contains a gene encoding the bFGF-GST fusion protein and a 5′ UTR. The gene can be transcribed using T7 RNA polymerase. The unique feature of the transcript lies in the 44-nt 5′UTR, which contains a small stem-loop element (MBR) consisting nucleotide +8 to +35 (FIG. 1). The sequence of MBR is derived from one of the RNA aptmers that showed highest affinity (Kd=0.17-0.21 nM) to human 18 kDa bFGF (Jellinek et al., Proc. Natl. Acad. Sci. USA 90: 11227-31 (1993)). The MBR also contains three base pairs (nucleotides +5 to +7 and +36 to +38) added to the stem of the original aptmer sequence to re-enforce the structure. The MBR is positioned only 4 nt away from the 5′-end of the transcript generated by transcribing pUFG with T7 polymerase, and 6 nt upstream of the start codon of the bFGF gene. The sequence immediately upstream of the start codon (nucleotides +39 to +44) is based on a ribosome binding sequence (RBS) optimized for translation in rabbit reticulocyte lysate (Ambion, TX). The pUFG plasmid is the construct that will be used for generating Random Sequence libraries, and also will be used in experiments described infra.

### Example II: Selection of a Hemaglutinin Tag Peptide

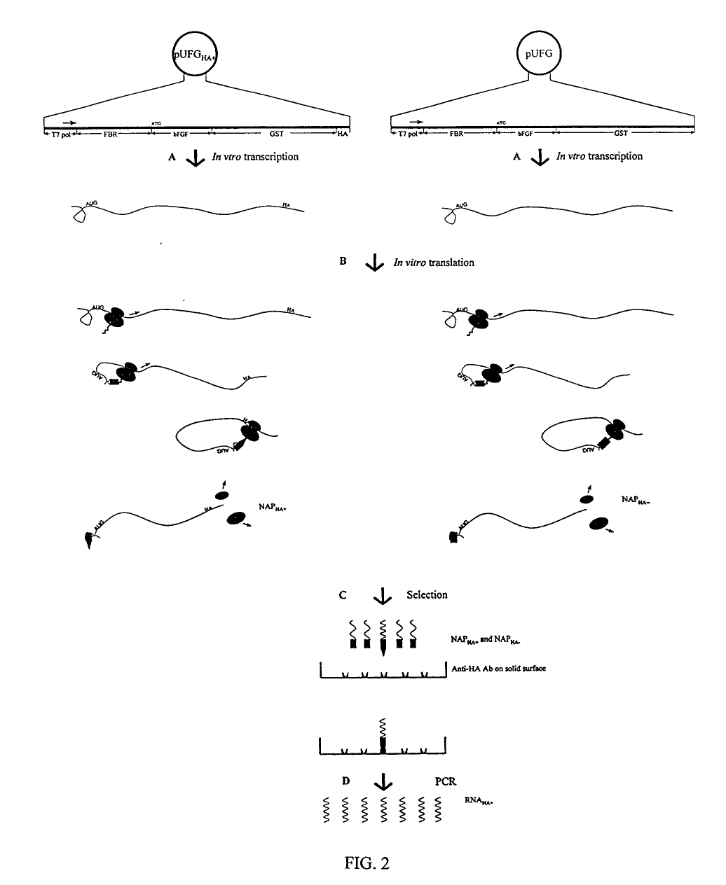

First, two almost-identical RNAs are generated, one encoding a peptide that contains a hemaglutinin (HA) tag (RNA_{HA+}) and the other encoding a polypeptide that lacks a hemaglutinin (HA) tag (RNA_{HA-}). Second, by using an anti-HA antibody that is attached to a solid surface and using ENTRAP (of the present invention), RNA_{HA+} can be selected and enriched in the pool while RNA_{HA -} depleted. The overall experiment procedures are illustrated in FIG. 2. The procedures consist of the following:

A) in vitro transcription that transcribes the fusion genes in plasmid pUFG_{HA+} and pUFG_{HA-} to RNA in separate reactions. The two RNAs have the same sequences except that one encodes an additional HA tag (RNA_{HA+}) at the C-terminal of protein while the other does not (RNA_{HA-}). Both RNAs contain a sequence (MBR) in the 5′-untranslated region (5′UTR) that specifically binds to basic fibroblast growth factor (bFGF) and a sequence that encodes the bFGF-GST fusion protein. Translation of these RNA's can produce complexes, herein termed Nucleic Acid Linked Peptides (NAPs), including an RNA_{HA+} and an RNA_{HA-}.

B) in vitro translation, equal amounts of the RNA_{HA+} and RNA_{HA -} will be translated in one reaction mixture.

C) NAP_{HA+} is subsequently immunoprecipitated using an anti-HA antibody and anti-mouse IgG coated magnetic beads.

D) enrichment and detection by reverse-transcription polymerase chain reaction (RT-PCR) by amplifying the cDNA from the RNA that is retained on the magnetic beads solid surface. When ENTRAP is successful and complete, only one band or a major band representing RNA_{HA+} will appear in the gel. However, if the method fails, two bands of equal intensity representing RNA_{HA+} and RNA_{HA-} will appear in the gel. The DNA band representing RNA_{HA+} will be sequenced. The ratio of input RNA_{HA+} to RNA_{HA-} can be decreased so that the former is a very minor fraction of the latter. This will test the ability of this method to select out sequences that encode peptides with the desired property from complex libraries.

Construction of Plasmids pUFG and pUFG_{HA}

The construction of plasmid pUFG is described in Example 1. Plasmid pUFG_{HA} is also synthesized as described in Example 1, but following step 2, the hemaglutinin tag sequence is added by the following method:

To introduce a nucleic acid sequence encoding HA tag to the 3′-end of the bFGF-GST gene, a pair of PCR primers, HA-F and HA-R (SEQ ID NO:7 and SEQ ID NO:8), are synthesized (Biogenesys, TX). PCR is carried out using the pair of primers, a DNA template that encodes three consecutive HA tag genes SEQ ID NO:9), and Taq DNA polymerase as commercially available (Innis, et al. (eds.), PCR protocols: A Guide to Methods and Applications, Academic Press, San Diego, Calif. (1989)). The PCR product, which is approx. 100 bp in size, is digested by EcoRI and XhoI (New England Biolabs, MA). The reaction is resolved in 2% agarose gel and the DNA is purified using NucleoTrap DNA extraction kit (Clontech, CA).

The plasmid pUFG is linearized using EcoRI and YhoI (New England Biolabs, Beverly, Mass.) and the linearized plasmid is purified from a 1% agarose gel as described previously. The purified HA DNA fragment and the linearized pUFG are ligated using T4 DNA ligase (New England Biolabs, Beverly, Mass.). The ligation reaction is used to transform competent E. coli DH5alpha. The transformant colonies are picked and expanded, and the plasmid pUFG_{HA} is isolated.

In vitro Transcription of Plasmids pUFG and pUFG_{HA}

Both plasmids are linearized in separate reactions using restriction enzyme BsaAI (New England Biolabs, Beverly, Mass.). The linearized plasmids are purified by phenol/chloroform extraction and isopropanol precipitation. Following isopropanol precipitation, the amount of each linearized plasmid is quantitated spectrophotometrically. One microgram of each linearized plasmid is added to separate transcription reactions using T7 RNA polymerase and the “maxi-prep” kit, and optionally including cap analog (m⁷G(5′)ppp(5′)G), according to the manufacturer's protocol (Ambion, TX). At the completion of the reactions, the reactions are treated with DNase I according to manufacturer's recommendations (Ambion, Austin, Tex.). The transcripts are precipitated with ammonium acetate and isopropanol, the pellet is washed twice with 70% ethanol and the pelleted transcripts are resuspended in 100 microL of distilled water. The amount of RNA produced in each reaction is determined by spectrophotometry by removing an aliquot from each and reading the absorbance at 260 and 280 nm.

In vitro Translation of pUFG and pUFG_{HA} Transcripts

One-tenth of a microgram of each of the RNAs are pooled and added to the reticulocyte lysate translation reaction mix with an RNase inhibitor (Ambion, TX) and the translation reaction is incubated at 30 ?C. for one hour.

NAP Selection Procedure

An appropriate amount of anti-HA monoclonal antibody (BAbCO, Richmond, Calif.) is added to the translation reaction. Following a 30 min. incubation, human anti-mouse IgG-coated magnetic beads (Dynal, Lake Success, N.Y.) is added to the mixture. The amount of beads added is sufficient to provide binding capacity in excess of the amount of primary anti-HA antibody that was added to the translation reaction. The tube is incubated 1 hour at 22 ?C. with constant inversion. The magnetic beads are collected by a magnetic stand. The beads are carefully washed with TBS/0.2M NaCl (pH 7.5) three times. Then the beads are washed twice in 5 mM Tris, pH 7.5. Finally the beads are resuspended in a minimal volume of 1× Reverse Transcription Buffer and cDNA is synthesized from the RNA template of the captured NAPs using MMLV Superscript reverse transcriptase (GIBCO BRL, Bethesda, Md.). The cDNA is amplified in a PCR reaction containing a pair of primers, GST-F459 and GST-end (SEQ ID NO:10 and SEQ ID NO:11) and commercially available Taq DNA polymerase. The beads are captured, and the PCR product which is released into solution is resolved in a 1.5% agarose gel. Since the anti-HA antibodies only capture NAP_{HA+} but not NAP_{HA-}, only the 620-bp PCR product (containing 3′-end of the GST and the entire HA tag gene sequence) should appear in the gel while the 512-bp PCR product (containing only the 3′-end of the GST gene sequence) should not appear (FIG. 3). In a parallel control experiment, where a polyclonal goat anti-GST antibody (Pharmacia, Piscataway, N.J.) is used instead of the anti-HA antibody, both the 620-bp and 512-bp PCR products should appear in the gel. The selection procedure should be able to select NAPHA+s even when they are a minor fraction of the NAP pool. Therefore, a pool of RNA transcripts with the ratio of pUFG_{HA} transcripts:pUFG transcripts of 1:10, 1:100, or 1:1,000 is added to the translation reaction, and the same selection and PCR reaction is performed and may be reiterated. In all these cases, the PCR product is either the 620-bp only or with a minor 512-bp band. The minor 512-bp may be caused by incomplete washing, and can be reduced by increasing the washing stringency in the selection procedure such that the binding of the bFGF RBD to the MBR of the transcripts is not disrupted, for example, by increasing the salt concentration (for wash buffers that do not disrupt high-affinity protein-RNA binding, see, for example Burke et al., J. Mol. Biol. 264: 650-666 (1996); Tuerk et al., J. Mol. Biol. 213: 749-761 (1990); see also U.S. Pat. No. 5,270,163 to Gold and Tuerk)). However, in the parallel experiments using goat anti-GST antibody, which captures both pUFG and pUFG_{HA} transcripts, the ratio of the 620-bp to the 512-bp PCR product should correspond to the ratio of the input RNA.

An alternative method to monitor the selection procedure is to use ³²P-labeled RNA transcripts. ³²P is incorporated by using a radiolabeled ribonucleotide in the transcription reaction. After the selection and washing procedure, the captured RNA can be recovered from of the NAPs that are attached to the beads using equal volumes of 50% urea and phenol, extracting the phenol with chloroform, and precipitating the RNA using carrier RNA (Burke et al., J. Mol. Biol. 264: 650-666 (1996)). The selected RNA is resolved in a high-resolution denaturing-polyacrylamide gel (about 4%) which is then dried and exposed to a phosphorimaging screen. The screen can be analyzed on a phosphorimager to quantitate the amount of each transcript captured in the selection procedure. The pUFG_{HA} transcript (approx. 1,400 nt) is 108 nt longer than the pUFG transcript, and thus the two can be distinguished on a gel. All or most of the transcript captured with the anti-HA antibody should be the pUFG transcript.

### Example III: Selection of a Peptide that Exhibits Ni²⁺-Dependent Binding to the Mouse IgG Fc Fragment

The purpose of this Example is to demonstrate that a specific target-binding NAP can be selected from a NAP library with randomized sequences using mouse antibody Fc fragment as the target molecule. The present method can be used to screen for peptides that bind mouse Fc in a nickel-dependent fashion. Such peptides would be extremely useful in the purification of monoclonal antibodies, so that removal of the monoclonal antibodies from a purification peptide matrix would only require removal of nickel. This would avoid the use of low pH buffers currently used to remove monoclonal antibodies from columns that use anti-Fc antibodies, protein A or protein G. Such buffers often destroy the secondary structure of the antibodies being purified.

Introducing a Randomized Nucleic Acid Sequence to the 3 ′-end of the bFGF-GST Gene

A stretch of randomized DNA sequence are added to the 3′-end of the fusion protein bFGF-GST gene. In order to do this, about one microgram of plasmid pUFG is linearized at the 3′-end of the bFGF-GST gene using EcoRI and XhoI restriction enzymes (New England Biolabs, Beverly, Mass.). Two oligonucleotides are synthesized and one microgram of each are annealed together (SEQ ID NO:12 and SEQ ID NO:13) to generate a double-stranded BglI linker with two sticky ends that are complementary to the EcoRI and XhoI sticky ends on the linearized plasmid. The linker is then ligated to the linearized plasmid to generate pUFG′, a modified pUFG that has a new BglI site at the 3′-end of the bFGF-GST gene. The pUFG′ is then used to transform E. coli DH5alpha and the plasmid is purified from the expanded transformant clone. The purified pUFG′ is linearized by BglI and XhoI, phenol/chloroform extracted, and isopropanol precipitated.

An oligonucleotide, RSOL (SEQ ID NO:14), consisting of a randomized sequence 60 nucleotides long bracketed by defined sequence, is synthesized. The oligonucleotide GST-end (SEQ ID NO:11) is annealed to RSOL and double-stranded DNA is synthesized using T4 DNA polymerase (New England Biolabs, MA). The double-stranded DNA is digested by DraIII to generate a sticky end that is complimentary to the BglI sticky end of the modified linearized pUFG′.

Without further purification, 1 picomole of pUFG′ (a linear molecule) and four picomoles of the double-stranded randomized sequence RSOL digested with DraIII are mixed and ligated by T4 DNA ligase (New England Biolabs, MA). The desired ligation reaction of RSOL at the DraIII site to pUFG′ at the BglI site does not regenerate either restriction site, and thus is not affected by the presence of these restriction enzymes. Because the vector cannot self-ligate (it has incompatible BglI and XhoI ends) and the RSOL cannot ligate tandemly to other RSOL molecules, so that by using this method, over 95% of the linearized vector can be ligated with one stretch of the randomized sequence to the 3′-end of the GST gene. The resulting linear molecules can be transcribed directly; there is no need to transform E. coli and isolate DNA, which can result in reduced complexity of the library.

Transcription of the Random Sequence library

One microgram of the DNA library is added to a transcription reaction using T7 RNA polymerase and the “maxi-prep” kit, and optionally including cap analog G(m⁷(5′)ppp(5′)G), according to manufacturer's recommendations (Ambion, TX). At the completion of the reactions, the reactions are treated with DNase I according to manufacturer's recommendations (Ambion, Austin, Tex.). The transcripts are precipitated with ammonium acetate and ethanol, the pellet is washed twice with 70% ethanol and the pelleted transcripts are resuspended in 100 microliter of distilled water. The amount of RNA produced in the reaction is determined by spectrophotometry by removing an aliquot from each and reading the absorbance at 260 and 280 nm. The transcription reaction is performed at 30° C. for 60 min.

Translation of the Random Sequence Library Transcripts

The purified RNA is added directly to the reticulocyte lysate translation reaction mix (Ambion, Austin, Tex.) in a final volume of 50 microliter and the translation reaction is incubated at 30 ?C. for one hour.

NAP Selection Procedure

Capture Using Mouse Fc as a Target Molecule in the Presence of Ni²⁺

To obtain a peptide that binds the Fc fragment of mouse immunoglobulins in a metal dependent fashion, mouse Fc fragments (Sigma, MO) will be conjugated to magnetic beads (DYNAL, NY) according to the manufacturer's protocol. The Fc-coated magnetic beads will be washed three times in 20 mM Tris-HCl/0.2M NaCl, pH 7.5 containing 50 mM Ni(Cl)₂. The entire translation mixture will be diluted into a buffer to bring the final volume to 1 mL with final Ni²⁺ concentration of 20 mM and Tris-HCl to 20 mM, NaCl to 0.2 M. The translation mix is added to a tube containing the magnetic beads and incubated at 22 ?C. for 2 hours with inversion. Following incubation, the magnetic beads are captured using a magnet, and the binding buffer is removed. The beads are washed three times with 20 mM Tris/0.2 M NaCl that lacks Ni(Cl)₂. To increase stringency, different wash buffers may be used that do not disrupt the MBR-RBD (bFGF) protein-RNA binding, as determined in Example II, part 4.

After washing, it is expected that only NAPs binding to the Fc in a manner that 1) tolerates Ni²⁺, or 2) requires Ni²⁺, will be present on the magnetic beads. The beads are washed three times in 5 mM Tris, pH 7.5. Finally the beads are resuspended in a minimal volume of 1× Reverse Transcription Buffer and cDNA is synthesized from the RNA template of the captured NAPs using MMLV Superscript reverse transcriptase (GIBCO BRL, MD) and the 3′ primer GST-end (SEQ ID NO:11). The cDNA is precipitated using glycogen as a carrier. The cDNA is amplified in a PCR reaction containing the GST-end and GST-aug primers (SEQ ID NO:11 and SEQ ID NO:15), and Taq DNA polymerase as commercially available. At the end of the PCR, the beads are captured, and an aliquot of the PCR is analyzed on a gel. The entire ENTRAP procedure may be reiterated; in each round the washing stringency may be increased. The final PCR products may be cloned into a plasmid vector and sequenced to identify the peptides encoded by the random sequences by their deduced amino acid sequences.

Determination of Ni²⁺ Dependence of Binding of Selected Peptides to Fc

Selected peptides that bind Fc in the presence of Ni²⁺ are synthesized based on their deduced amino acid sequences. The peptides are conjugated to BSA using methods known in the art (Wong, Chemistry of Protein Conjugation and Cross-Linking, CRC Press, Boca Raton (1993)). The BSA-conjugated peptides are absorbed onto the wells of duplicate 96-well plates (Nunc-Immuno Plate MaxiSorp™, Nunc, Denmark). TBS containing 50 mM Ni(Cl)₂ is added to the wells of one plate, and TBS lacking Ni(Cl)₂ is added to wells of the duplicate plate. The binding of mouse Fc to the identified peptides in the presence and absence of Ni²⁺ is determined by ELISA (Bost et al., Proc. Natl. Acad. Sci. USA 82: 1372-1375 (1985)) using goat anti-mouse IgG conjugated to alkaline phosphatase and p-nitrophenyl phosphate as a detection reagent. Selected peptides that bind Fc in the presence, but not in the absence, of Ni²⁺ can be futher tested in the multi-well format to determine whether, after allowing Fc to bind, binding can be disrupted by washing in buffers lacking Ni²⁺ and subsequently washing in buffers containing 10 mM EDTA. Identified peptides that bind Fc in a Ni²⁺-dependent fashion, and release Fc when Ni²⁺ is removed, can be developed as purification reagents for monoclonal antibodies.

The ELISA format can also be used to determine the binding affinities of identified peptides. Absorbance values which rely on the detection of bound Fc are compared with a standard curve for Fc concentration to determine the amount of bound and unbound Fc in the assays (Bost et al., supra). Kds for peptide-Fc binding in the presence and absence of Ni²⁺ can be determined by the method of Scatchard (Ann. N.Y. Acad. Sci. 51: 660-(1949)), as modified by Munson and Rodbard (Anal. Biochem. 107: 220-239 (1980)) using the LIGAND program. Peptides that bind Fc in the presence, but not in the absence, of Ni²⁺ and dissociate from the Fc when EDTA is added, can be linked to a matrix, such as a chromatography matrix, and used to purify mouse monoclonal antibodies.

All publications, including patent documents, scientific articles and www sites, referred to in this application are incorporated by reference in their entirety for all purposes to the same extent as if each individual publication were individually incorporated by reference.

All headings are for the convenience of the reader and should not be used to limit the meaning of the text that follows the heading, unless so specified.

### Nucleic Acid Sequences with Annotations

SEQ ID NO:1

Human
FGF-aug
GCGAAGCTTATATAAGGTACCAGGAGGTGAACCATGGCAGCCGGGA
HindIII, KpnI, and NcoI sites are underlined, re-
spectively; gene-specific sequence is in italic.

SEQ ID NO:2

Human
FGF-end
GCGTCTAGATAGTCCAGGGCCCTGAAAATACAGGTTTTCGCTCTTAGCAG
ACATTGGAAGAA
XbaI site is underlined; gene-specific sequence is
in italic.

SEQ ID NO:3

Synthetic
GEX-F285
CGCTCTAGACTAGGTTATTGGAAAA
XbaI site is underlined.

SEQ ID NO:4

Synthetic
GEX-R239
CGCAAGCTTACTGTTTCCTGTGTG
HindIII site is underlined.

SEQ ID NO:5

T7 Bacteriophage
T7-MBR1
AGTGGTACCTAATACGACTCACTATAGGAGCTCGAAGG
KpnI and SacI sites are underlined, respectively.

SEQ ID NO:6

T7 Bacteriophage
T7-MBR2
TCACCATGGTGGCCTCGAAGTGTGCTTGCCTATACGTTGCCTTCGAGCTC
CT
NcoI and SacI sites are underlined, respectively.

SEQ ID NO:7

Influenza Virus
HA-F
CCAGAATTCTACCCATACGATGTTCC
EcoRI site is underlined.

SEQ ID NO:8

Influenza Virus
HA-R
TGCCTCGAGCTAGCACTGAGCAGCGT
XhoI site is underlined.

SEQ ID NO:9

Influenza Virus
The DNA template that encodes three consecutive HA
genes
TTTTACCCATACGATGTTCCTGACTATGCGGGCTATCCCTATGACGTCCC
GGACTATGCAGGATCCTATCCATATGACGTTCCAGATTACGCTGCTCAGT
GCTAG
Primer binding regions are underlined.

SEQ ID NO:10

Synthetic
GST-F459
TCTATGGCCATCATACGTT

SEQ ID NO:11

Synthetic
GST-END
GAGGCAGATCGTCAGTCA

SEQ ID NO:12 and SEQ ID NO:13

Synthetic
BglI linker
^{5′}AATTCGCCAGGCAGGC
GCGGTCCGTCCGAGCT^{5′}
BglI restriction site is underlined.

SEQ ID NO:14

Synthetic
RSOL
ATACACGGCGTGGTCTTGCAATA(NN...NN)₆₀ TGACTGACGATCTGC
CTC
DraIII site is underlined, the sequence comple-
mentary to the GST-END is in italic.

SEQ ID NO:15

Synthetic
RSF
ATACACGGCGTGGTCTTGCAATA
DraIII site is underlined.

What is claimed is:

1. A nucleic acid molecule, comprising a nucleotide sequence that:
a) comprises a moiety binding region; and
b) encodes an interacting domain,
wherein said interacting domain directly or indirectly binds with said moiety binding region.

2. The nucleic acid molecule of claim 1, wherein said nucleic acid molecule comprises ssRNA or dsRNA.

3. The nucleic acid molecule of claim 2, further comprising or encoding at least one random sequence or at least one sequence of interest.

4. The nucleic acid molecule of claim 1, wherein said nucleic acid molecule comprises ssDNA.

5. The nucleic acid molecule of claim 4, further comprising or encoding at least one random sequence or at least one sequence of interest.

6. The nucleic acid molecule of claim 1, wherein said nucleic acid molecule comprises dsDNA.

7. The nucleic acid molecule of claim 6, further comprising or encoding at least one random sequence or at least one sequence of interest.

8. The nucleic acid molecule of claim 1, further comprising or encoding a spacer region.

9. The nucleic acid molecule of claim 8, wherein said spacer region comprises or encodes at least one purification domain or at least one detection domain.

10. The nucleic acid molecule of claim 1, further comprising or encoding at least one expression control sequence.

11. The nucleic acid molecule of claim 1, further comprising or encoding at least one start codon.

12. A vector comprising the nucleic acid molecule of one of claims 1, 2, 4 or 6.

13. A vector comprising the nucleic acid molecule of one of claims 3, 5 or 7.

14. The vector of one of claims 12 or 13, wherein said vector is selected from the group consisting of a viral vector, a plasmid, a phage, a liposome, a microsphere or a linear dsDNA molecule.

15. The nucleic acid molecule of one of claims 1, 2, 4 or 6, wherein said nucleic acid molecule is operably linked to said interacting domain.

16. The nucleic acid molecule of one of claims 3, 5 or 7, wherein said nucleic acid molecule is operably linked to said interacting domain.

17. The nucleic acid molecule of one of claims 15 or 16, wherein said interacting domain binds directly or indirectly with said moiety binding region.

18. The nucleic acid molecule of one of claims 2 or 3, wherein said nucleic acid molecule is operably linked to said interacting domain.

19. The nucleic acid molecule of one of claims 3, 5 or 7 wherein said nucleic acid molecule is operably linked to a polypeptide encoded by said random sequence or said sequence of interest.

20. The nucleic acid molecule of claim 18, wherein said nucleic acid molecule is substantially devoid or devoid of ribosomes.

21. The nucleic acid molecule of one of claims 1, 2, 3, 4, 5, 6 or 7, wherein said moiety binding region is substantially free of secondary structure.

22. The nucleic acid molecule of one of claims 3, 5 or 7, wherein said moiety binding region is substantially free of secondary structure.

23. The nucleic acid molecule of any one of claims 21 or 22, wherein said moiety binding region directly or indirectly reduces the efficiency of translation of said nucleic acid molecule.

24. The nucleic acid molecule of one of claims 1, 2, 3, 4, 5, 6 or 7, wherein said moiety binding region comprises at least one secondary structure.

25. The nucleic acid molecule of one of claims 3, 5 or 7, wherein said moiety binding region comprises at least one secondary structure.

26. The nucleic acid molecule of one of claims 22, 23, 24 or 25, wherein said at least one secondary structure is a stem-loop configuration or a hairpin configuration or wherein two stretches of complementary sequences are in one nucleic acid molecule.

27. The nucleic acid molecule of one of claims 24, 25 or 26, wherein said secondary structure is within between about 60 nucleotides and about 2 nucleotides of a start codon, within between about 50 nucleotides and about 4 nucleotides of a start codon, within between about 40 nucleotides and about 6 nucleotides of a start codon, within between about 30 nucleotides and about 8 nucleotides of a start codon or within between about 20 nucleotides and about 10 nucleotides of a start codon.

28. The nucleic acid molecule of one of claims 24, 25, 26 or 27 wherein said secondary structure directly or indirectly reduces the efficiency of translation of said nucleic acid molecule, optionally by the pairing of said two stretches of complementary sequences are in one nucleic acid molecule.

29. The nucleic acid molecule of one of claims 15, 16, 17 or 18, wherein said moiety binding region binds with said interacting domain to form a moiety binding region/interacting domain complex.

30. The nucleic acid molecule of claim 16, wherein said moiety binding region is operably linked to said interacting domain to form a moiety binding region/interacting domain complex.

31. The nucleic acid molecule of any one of claims 29 or 30, wherein said moiety binding region/interacting domain complex reduces the efficiency of translation of said nucleic acid molecule.

32. The nucleic acid molecule of any one of claims 29 or 30, wherein said interacting domain comprises a polypeptide.

33. The nucleic acid molecule of one of claims 15, 16, 17 or 18, wherein said polypeptide encoded by said random sequence is bound with a substance of interest.

34. The nucleic acid molecule of claim 33, wherein said substance of interest is on a solid support.

35. The nucleic acid molecule of claim 33, wherein said substance of interest is on or within a cell.

36. The nucleic acid molecule of claim 35, wherein said cell is ex vivo.

37. The nucleic acid molecule of claim 35, wherein said cell is in vivo in a subject.

38. The nucleic acid molecule of claim 35, wherein said cell is a normal cell or an abnormal cell.

39. The nucleic acid molecule of claim 38, wherein said abnormal cell is a neoplastic cell or a virus infected cell.

40. The nucleic acid molecule of claim 33, wherein said substance of interest is on or within an etiological agent.

41. The nucleic acid molecule of claim 40, wherein said etiological agent is selected from the group consisting of a bacteria, a spore, a virus, a parasite or a prion.

42. The nucleic acid molecule of claim 33, wherein said substance of interest comprises at least one organic molecule, an inorganic molecule, a polymer, a polypeptide, a nucleic acid molecule, a ribozyme, a lipid, a carbohydrate, a small molecule, a biomacromolecule or a drug.

43. A library of nucleic acid molecules of one of claims 3, 5 or 7.

44. The library of nucleic acid molecules of claim 43, wherein said library comprises at least two different random sequences, at least two different sequences of interest or a combination of at least one random sequence and at least one sequence of interest.

45. A library of nucleic acid molecules of claim 16.

46. The library of nucleic acid molecules of claim 45, wherein said library comprises at least two different random sequences, at least two different sequences of interest or a combination of at least one random sequence and at least one sequence of interest.

47. A library of nucleic acid molecules of one of claims 19 or 20.

48. The library of nucleic acid molecules of claim 47, wherein said library comprises at least two different random sequences, at least two different sequences of interest or a combination of at least one random sequence and at least one sequence of interest.

49. A library of nucleic acid molecules of one of claims 21 or 25.

50. The library of nucleic acid molecules of claim 49, wherein said library comprises at least two different random sequences, at least two different sequences of interest or a combination of at least one random sequence and at least one sequence of interest.

51. A library of nucleic acid molecules of claim 30.

52. The library of nucleic acid molecules of claim 51, wherein said library comprises at least two different random sequences, at least two different sequences of interest or a combination of at least one random sequence and at least one sequence of interest.

53. The library of nucleic acid molecules of any one of claims 42 to 55, wherein said library is contacted with at least one substance of interest.

54. The library of nucleic acid molecules of claim 53, wherein said at least one substance of interest is directly or indirectly bound on a solid support or in solution.

55. The nucleic acid molecule of claim 53, wherein said substance of interest is on or within a cell.

56. The nucleic acid molecule of claim 55, wherein said cell is ex vivo.

57. The nucleic acid molecule of claim 55, wherein said cell is in vivo in a subject.

58. The nucleic acid molecule of claim 55, wherein said cell is a normal cell or an abnormal cell.

59. The nucleic acid molecule of claim 58, wherein said abnormal cell is a neoplastic cell or a virus infected cell.

60. The nucleic acid molecule of claim 53, wherein said substance of interest is on or within an etiological agent.

61. The nucleic acid molecule of claim 60, wherein said etiological agent is selected from the group consisting of a bacteria, a virus, a parasite or a prion.

62. A library of vectors of one of claims 12, 13 or 14.

63. A method for identifying a nucleic acid molecule or sequence, comprising:
1. providing at least one nucleic acid molecule of claim 3 or claim 5;
2. translating said nucleic acid molecule to provide at least one complex, wherein said complex comprises a polypeptide operably linked to a random sequence or a nucleic acid sequence or a nucleic acid molecule of interest;
3. contacting said at least one complex with at least one substance of interest;
4. selecting at least one complex that binds with said at least one substance of interest; and
5. identifying said random sequence or said nucleic acid sequence of interest or nucleic acid molecule of interest.

64. The method of claim 63, wherein said contacting comprises conditions that promote binding of said complex to said substance of interest.

65. The method of claim 63, wherein said identifying comprising amplifying said random sequence or said nucleic acid sequence of interest or said nucleic acid molecule of interest.

66. The method of claim 63, wherein said substance of interest is on a solid support or in solution.

67. The method of claim 63, wherein said substance of interest is on or within a cell.

68. The method of claim 63, wherein said substance of interest is on or within an etiological agent.

69. A nucleic acid molecule comprising a random sequence or nucleic acid sequence or nucleic acid molecule identified by the method of claim 63.

70. The method of claim 63, further comprising the step of sequencing the identified random sequence or nucleic acid sequence of interest or said nucleic acid molecule of interest.

71. The method of claim 63, further comprising performing steps 1, 2, 3 and 4 reiteratively.

72. The method of claim 63, further comprising performing steps 1, 2, 3, 4 and 5 reiteratively.

73. A method for identifying a nucleic acid molecule or sequence, comprising:
1. providing at least one nucleic acid molecule of one of claims 5 or 7;
2. transcribing said nucleic acid molecule to a corresponding RNA molecule;
3. translating said RNA molecule to provide at least one complex, wherein said complex comprises a polypeptide operably linked to a random sequence or a nucleic acid sequence of interest or a nucleic acid molecule of interest;
4. contacting said at least one complex with at least one substance of interest;
5. selecting at least one complex that binds with said at least one substance of interest; and
6. identifying said random sequence or nucleic acid sequence of interest or nucleic acid molecule of interest.

74. The method of claim 73, wherein said contacting comprises conditions that promote binding of said complex to said substance of interest.

75. The method of claim 73, wherein said identifying comprising amplifying said random sequence or nucleic acid sequence of interest or nucleic acid molecule of interest.

76. The method of claim 73, wherein said substance of interest is on a solid support or in solution.

77. The method of claim 73, wherein said substance of interest is on or within a cell.

78. The method of claim 73, wherein said substance of interest is on or within an etiological agent.

79. A nucleic acid molecule comprising a random sequence or nucleic acid sequence or nucleic acid molecule identified by the method of claim 73.

80. The method of claim 73, further comprising the step of sequencing the identified random sequence or nucleic acid sequence of interest or said nucleic acid molecule of interest.

81. The method of claim 73, further comprising performing steps 1, 2, 3 and 4 reiteratively.

82. The method of claim 73, further comprising performing steps 1, 2, 3, 4 and 5 reiteratively.

83. The method of claim 73, further comprising performing steps 1, 2, 3, 4, 5 and 6 reiteratively.

84. A method for identifying a polypeptide encoded by a random nucleic acid sequence or nucleic acid sequence of interest or nucleic acid molecule of interest, comprising:
1. providing at least one nucleic acid molecule of claim 3 or 5;
2. translating said nucleic acid molecule to provide at least one complex, wherein said complex comprises a polypeptide operably linked to a random sequence or a nucleic acid sequence of interest or a nucleic acid molecule of interest;
3. contacting said at least one complex with at least one substance of interest;
4. selecting at least one complex that binds with said at least one substance of interest; and
5. identifying said polypeptide in said complex.

85. The method of claim 84, wherein said contacting comprises conditions that promote binding of said transcript to said substance of interest.

86. The method of claim 84, wherein said identifying comprises amplifying said random sequence or nucleic acid sequence of interest or nucleic acid molecule of interest or nucleic acid molecule of interest.

87. The method of claim 84, wherein said substance of interest is on a solid support or in solution.

88. The method of claim 84, wherein said substance of interest is on or within a cell.

89. The method of claim 84, wherein said substance of interest is on or within an etiological agent.

90. A polypeptide identified by the method of claim 84.

91. The method of claim 84, further comprising the step of sequencing said identified polyeptide.

92. The method of claim 84, further comprising performing steps 1, 2, 3 and 4 reiteratively.

93. The method of claim 84, further comprising performing steps 1, 2, 3, 4 and 5 reiteratively.

94. A method for identifying a polypeptide encoded by a random nucleic acid sequence or nucleic acid sequence of interest or nucleic acid molecule of interest in the nucleic acid, comprising:
1. providing at least one nucleic acid molecule of one of claims 5 or 7;
2. transcribing said nucleic acid molecule to a corresponding RNA molecule;
3. translating said RNA molecule to provide at least one complex, wherein said complex comprises a polypeptide operably linked to a random sequence or a nucleic acid sequence of interest or a nucleic acid molecule of interest;
4. contacting said at least one complex with at least one substance of interest;
5. selecting at least one complex that binds with said at least one substance of interest; and
6. identifying said polypeptide in said complex.

95. The method of claim 94, wherein said contacting comprises conditions that promote binding of said transcript to said substance of interest.

96. The method of claim 94, wherein said identifying comprising amplifying said random sequence or nucleic acid sequence of interest or nucleic acid molecule of interest or nucleic acid molecule of interest.

97. The method of claim 94, wherein said substance of interest is on a solid support or in solution.

98. The method of claim 94, wherein said substance of interest is on or within a cell.

99. The method of claim 94, wherein said substance of interest is on or within an etiological agent.

100. A polypeptide identified by the method of claim 94.

101. The method of claim 94, further comprising the step of sequencing the identified polypeptide.

102. The method of claim 94, further comprising performing steps 1, 2, 3 and 4 reiteratively.

103. The method of claim 94, further comprising performing steps 1, 2, 3, 4 and 5 reiteratively.

104. The method of claim 94, further comprising performing steps 1, 2, 3, 4, 5 and 6 reiteratively.

105. A method for identifying a test compound, comprising:
a) contacting a target with a complex that:
1) comprises a moiety binding region;
2) encodes an interacting domain; and
3) comprises a random sequence or a sequence of interest that encodes a polypeptide;
wherein said interacting domain directly or indirectly binds with said moiety binding region;
b) identifying polypeptides bound with said target;
c) determining the structure of said polypeptide; and
d) identifying moieties that have structures that have space filling shapes that are similar to at least a portion of said polypeptide.

106. A test compound identified by method of claim 105.

107. A pharmaceutical composition identified by a method of claim 105.

108. A method for identifying a target, comprising:
a) contacting a substance of interest with a complex that:
1) comprises a moiety binding region;
2) encodes an interacting domain; and
3) comprises a random sequence or a sequence of interest that encodes a polypeptide;
wherein said interacting domain directly or indirectly binds with said moiety binding region;
b) identifying targets that bind with said complex.

109. A target identified by the method of claim 108.

110. A pharmaceutical target identified by the method of claim 108.


SEQUENCE LISTING (15 sequences)

SEQ ID NO: 1 - DNA, 46 bp, Homo sapiens
gcgaagcttatataaggtaccaggaggtgaaccatggcagccggga

SEQ ID NO: 2 - DNA, 60 bp, Homo sapiens
gcgtctagatagtccagggccctgaaaatacaggttttcgctcttagcagacattggaag

SEQ ID NO: 3 - DNA, 25 bp, Artificial Sequence
  Synthetic Sequence
cgctctagactaggttattggaaaa

SEQ ID NO: 4 - DNA, 24 bp, Artificial Sequence
  Synthetic Sequence
cgcaagcttactgtttcctgtgtg

SEQ ID NO: 5 - DNA, 38 bp, Bacteriophage T7
agtggtacctaatacgactcactataggagctcgaagg

SEQ ID NO: 6 - DNA, 52 bp, Bacteriophage T7
tcaccatggtggcctcgaagtgtgcttgcctatacgttgccttcgagctcct

SEQ ID NO: 7 - DNA, 26 bp, Influenza virus
ccagaattctacccatacgatgttcc

SEQ ID NO: 8 - DNA, 26 bp, Influenza virus
tgcctcgagctagcactgagcagcgt

SEQ ID NO: 9 - DNA, 105 bp, Influenza virus
ttttacccatacgatgttcctgactatgcgggctatccctatgacgtcccggactatgcaggatcctatccatatgacgttccagattacgctgctcagtgctag

SEQ ID NO: 10 - DNA, 19 bp, Artificial Sequence
  Synthetic Sequence
tctatggccatcatacgtt

SEQ ID NO: 11 - DNA, 18 bp, Artificial Sequence
  Synthetic Sequence
gaggcagatcgtcagtca

SEQ ID NO: 12 - DNA, 16 bp, Artificial Sequence
  Synthetic Sequence
aattcgccaggcaggc

SEQ ID NO: 13 - DNA, 16 bp, Artificial Sequence
  Synthetic Sequence
tcgagcctgcctggcg

SEQ ID NO: 14 - DNA, 101 bp, Artificial sequence
  Synthetic Sequence
atacacggcgtggtcttgcaatannnnnnnnnnnnnnnnnnnnnnnnnnnnnnnnnnnnnnnnnnnnnnnnnnnnnnnnnnnntgactgacgatctgcctc

SEQ ID NO: 15 - DNA, 23 bp, Artificial Sequence
  Synthetic Sequence
atacacggcgtggtcttgcaata